(19) United States
(12) Patent Application Publication
(10) Pub. No.: US 2010/0298217 A1
(43) Pub. Date: Nov. 25, 2010

(54) CSF-1R MUTANTS

(76) Inventors: Evan Richard Stanley, New York, NY (US); Ying Xiong, Mount Pleasant, SC (US)

Correspondence Address: AMSTER, ROTHSTEIN & EBENSTEIN LLP, 90 PARK AVENUE, NEW YORK, NY, 10016, US

(21) Appl. No.: 12/451,680

(22) PCT Filed: May 22, 2008
(86) PCT No.: PCT/US2008/006533

§ 371 Date: Jun. 30, 2010

Related U.S. Application Data
(60) Provisional application No. 60/932,325, filed on May 30, 2007.

(51) Int. Cl. A61K 38/17; C12N 9/12; C07K 14/705; C12N 5/078; C12N 5/02; A61P 35/00; A61P 29/00; A61P 37/02; A61P 25/28; A61P 25/00; A61P 9/10; A61P 3/04

(52) U.S. Cl. 514/7.5; 435/194; 530/350; 435/325; 435/385

(57) ABSTRACT

Provided are mutant class III receptor tyrosine kinases (RTKIII) comprising a mutation at the amino acid residue corresponding to the conserved cysteine at residue 432 (C432) or 439 (C439) of a mouse colony-stimulating factor (1) receptor (CSF-IR) precursor having the amino acid sequence of SEQ ID NO: 1, where the mutation is a replacement of the cysteine with an amino acid having an uncharged polar R group. Also provided are mutant RTKIIIs comprising a tyrosine to phenylalanine mutation. Additionally provided are extracellular domains of the above-identified mutant RTKIIIs comprising cysteine to serine mutations. Further provided are stable cell lines of macrophages lacking a native CSF-IR. Also provided are isolated nucleic acids encoding any of the above-described mutant RTKIIIs or extracellular domains. Vectors comprising those nucleic acids are also provided. Additionally provided are methods of preparing the above-identified stable cell lines. Methods of treating a mammal comprising administering the above described extracellular domain to the mammal are also provided.

### CROSS-REFERENCE TO RELATED APPLICATIONS

This application claims the benefit of U.S. Provisional Patent Application No. 60/932,325 filed on May 30, 2007, the contents of which are hereby incorporated by reference.

### STATEMENT REGARDING FEDERALLY SPONSORED RESEARCH OR DEVELOPMENT

This invention was supported by NIH Grant No. CA26504-29. As such, the U.S. Government has certain rights in this invention.

### BACKGROUND OF THE INVENTION

The present invention generally relates to enzyme mutants. More specifically, the invention is directed to mutants of class III receptor tyrosine kinases having an apparent decreased dissociation rate from its cytokine ligand.

### SUMMARY OF THE INVENTION

The inventors have discovered that certain mutations in class III receptor tyrosine kinases (RTKIII) confer advantageous properties to the RTKIII, including, with some of the mutations, an apparent decreased dissociation rate from its cytokine ligand.

Thus, the invention is directed to a mutant class III receptor tyrosine kinase (RTKIII) comprising a mutation at the amino acid residue corresponding to the conserved cysteine at residue 432 (C432) or 439 (C439) of a mouse colony-stimulating factor 1 receptor (CSF-1R) precursor having the amino acid sequence of SEQ ID NO:1, where the mutation is a replacement of the cysteine with an amino acid having an uncharged polar R group.

Additionally, the invention is directed to an extracellular domain of the above-identified mutant RTKIII comprising cysteine to serine mutations.

The invention is further directed to a stable cell line of macrophages lacking a native CSF-1R.

Also, the invention is directed to isolated nucleic acids encoding any of the above-described mutant RTKIIIs or extracellular domains.

The invention is additionally directed to a vector comprising the above isolated nucleic acid and additional genetic elements allowing transformation, expression and secretion of the mutant RTKIII or extracellular domain in a mammalian cell.

Further, the invention is directed to a method of preparing a stable cell line of macrophages from a mammal where the stable cell line maintains CSF-1 responsiveness. The method comprises: isolate macrophages from the mammal; culture the macrophages in growth medium comprising CSF-1; immortalize the macrophages using an SV-U19-5 retrovirus; single cell plate the macrophages to create a cloned cell line; and culture the cloned cell line to create the stable cell line.

The invention is also directed to a method of preparing the stable cell line described above lacking a native CSF-1R. The method comprises: isolate macrophages from a Csf1r-/Csf1r- mammal; culture the macrophages in growth medium comprising granulocyte-macrophage colony stimulating factor, immortalize the macrophages; single cell plate the macrophages to create a cloned cell line; and culture the cloned cell line to create the stable cell line.

Additionally, the invention is directed to a method of treating a mammal having or at risk for undesirable activation of a native RTKIII. The method comprises administering to the mammal the above described extracellular domain, where the extracellular domain is from the same type of RTKIII as the native RTKIII.

### BRIEF DESCRIPTION OF THE DRAWINGS

FIG. 1 is graphs, micrographs and photographs of western blots describing the CSF-1R-deficient macrophage (MacCsf1r−/− (M−/−)) cell line system for examining the structure function of the CSF-1R. Bone marrow derived macrophages from Csf1r−/− and wild mice were immortalized to create M−/− and M+/+macrophage cell lines. CSF-1R and CSF-1R mutants can be expressed in M−/− cells by retroviral transduction using the MSCViresGFP retrovirus. M−/− and M−/−.Csf1r can be maintained in GM-CSF in which they exhibit a low degree of macrophage differentiation. Panel a is a graph showing the growth of M−/− cells in different concentrations of GM-CSF (relative concentrations indicated). Panel b is a graph showing growth of M−/− and M−/−.Csf1r macrophages for 7 days under with the indicated growth factors (starting cell number as in a). Panel c is phase contrast photomicrographs of M−/− and M−/−.Csf1r macrophages cultured in GM-CSF and CSF-1. Panel d is an FACScan analysis of the cell surface expression of the CSF-1 receptor on M−/−, M+/+ and M−/−.Csf1r macrophages. Panel e shows CSF-1R expression by western blot and phase contrast morphology on M−/−, M+/+ and M−/−.Csf1r macrophages. Panel f shows a FACScan analysis of the expression of the macrophage marker Mac1. Panel g shows an anti-phosphotyrosine western blot of cell lysates of M+/+ and M−/−.Csf1r macrophages.

FIG. 2 is graphs showing cell surface expression of the CSF-1R and CSF-1 stimulated cell proliferation of M−/− cells expressing CSF-1R tyrosine to phenylalanine (Y->F) mutations of the known tyrosine phosphorylation sites. Panel a shows the Y->F mutant CSF-1Rs. Panel b shows a FACScan analysis of the cell surface CSF-1R. Panel c shows proliferation of M−/−.CSF1R Y->F mutant and control (M−/−.WT_CSF-1R and M−/−.vector) cells.

FIG. 3 is photographs of western blots showing expression of mature and immature CSF-1Rs in M−/− cells expressing the WT CSF-1R and various extracellular domain (ECD) mutant CSF-1Rs.

FIG. 4 is a graph showing expression by FACS of the cell surface CSF-1Rs on M−/− cells expressing the WT CSF-1R and various ECD mutant CSF-1Rs.

FIG. 5 is graphs showing differentiation (Mac 1 positivity) of M−/− cells expressing the WT CSF-1R and various ECD mutant CSF-1Rs.

FIG. 6 is micrographs showing that M−/−.C439SCSF-1R and M−/−.C432/439SCSF-1R cells exhibit a less polarized morphology than M−/−.WTCSF-1R cells when cultured in the presence of human CSF-1.

FIG. 7 is micrographs showing that M−/−.C439SCSF-1R and particularly M−/−.C432/439SCSF-1R cells are more spread with fewer filopodia than M−/−.WTCSF-1R cells when cultured in the presence of human CSF-1.

FIG. 8 is micrographs showing that M−/−. C432SCSF-1R, M−/−.C439SCSF-1R and M−/−.C432/439SCSF-1R cells are more resistant to rounding up at 24 h after removal of CSF-1 than M−/−.WTCSF-1R cells.

FIG. 9 is micrographs showing that, compared with M−/−.WTCSF-1R cells, M−/−.C432/439SCSF-1R cells exhibit prolonged survival after removal of human CSF-1.

FIG. 10 is graphs showing that, compared with M−/−.WTCSF-1R and M−/−.C432/439ACSF-1R cells, which die, M−/−.C432/439SCSF-1R cells proliferate when cultured in the absence of human CSF-1.

FIG. 11 is photographs of western blots showing that, following addition of human CSF-1 to cells preincubated in the absence of CSF-1 for 20 h, M−/−.C432/439SCSF-1R cells, but not M−/−.C432/439ACSF-1R cells, exhibit dramatically decreased CSF-1R tyrosine phosphorylation, CSF-1R ubiquitination and decreased CSF-1R degradation compared with M−/−.WTCSF-1R cells.

FIG. 12 is a graph showing the slower internalization of M−/−.C432/439SCSF-1Rs, than M−/−.WTCSF-1Rs following addition of human CSF-1 to cells preincubated in the absence of CSF-1 for 20 h. In this experiment, after CSF-1R upregulation and prior to the addition of CSF-1, the level of expression of the mutant CSF-1R was approximately 70% of the level of expression of the WT CSF-1R.

FIG. 13 is graphs showing that, in contrast to the absence of significant CSF-1 binding on the surface of M−/−.WTCSF-1R cells at 24 h after removal of human CSF-1, CSF-1 binding to the surface of M−/−.C432/439SCSF-1R cells persists, indicating that M−/−.C432/439SCSF-1R cells have a very slow off-rate.

FIG. 14 is a diagram and a graph showing that the initial rates of binding of CSF-1 to upregulated M−/−.WTCSF-1R and M−/−.C432/439SCSF-1R cells are indistinguishable. In this experiment, after CSF-1R upregulation and prior to the addition of CSF-1, the maximum CSF-1 binding to the mutant CSF-1R was approximately 70% of the level of binding to the WT CSF-1R.

FIG. 15 shows the conservation of C432 and C439 among other class III receptor tyrosine kinases.

### DETAILED DESCRIPTION OF THE INVENTION

The inventors have discovered that certain mutations in class III receptor tyrosine kinases (RTKIII) confer advantageous properties to the RTKIII, including, with some of the mutations, an apparent decreased dissociation rate from its cytokine ligand. See Examples.

As discussed in the Examples, replacement of the cysteine with a serine, but not an alanine, provides the apparent decreased dissociation rate. Without being bound to any particular mechanism, it is believed that the conformation change provided by the serine leads to the decreased dissociation rate. Any other amino acid having an uncharged polar R group would be expected to provide the same conformation change. Any amino acid having an uncharged polar R group would be expected to allow binding and provide a decreased dissociation rate. Preferably, the replacement amino acid is a serine, a threonine, a tyrosine, an asparagine or a glutamine, since those are naturally occurring amino acids having uncharged polar R groups. Most preferably, the replacement amino acid is a serine.

An RTKIII is as defined in IPR001824, a protein with an extracellular ligand-binding region, a single transmembrane region and a cytoplasmic kinase domain, where the extracellular region has five to seven immunoglobulin-like domains and the middle of the kinase domain has a stretch of 70-100 hydrophilic residues. Examples of RTKIIIs are CSF-1R, platelet-derived growth factor receptor (PDGF-R), stem cell factor receptor (SCFR), vascular endothelial growth factor receptor (VEGF-R), and Flt ligand (FL) cytokine receptor Flk-2/Flt-3.

As used herein, a mutant RTKIII having the amino acid sequence of SEQ ID NO:1 or SEQ ID NO:2 can be lacking the signal peptide present in SEQ ID NO:1 and SEQ ID NO:2, which is residues 1-19 of both sequences. Such a mutant RTKIII lacking the signal peptide would be considered herein to be 100% homologous to SEQ ID NO:1 or SEQ ID NO:2.

The skilled artisan could determine the corresponding residues to residues 432 and 439 of SEQ ID NO:1 for any RTKIII by simply comparing the sequence of that RTKIII with SEQ ID NO:1 and identifying the conserved cysteines from the RTKIII at a similar region of the protein.

These invention mutants include those where the mutation is C432S (including those that correspond to C432S), with or without any other mutations, including C439S (including those that correspond to C439S). Similarly included are those where the mutation is C439S, with our without any other mutations, including C432S. Also included are mutations that comprise both C432S and C439S mutations (including those that correspond to C432S and C439S).

As established in the Examples, these mutants preferably have a delayed dissociation rate from their cytokine ligand when compared to the unmutated receptor. Without being bound to any particular mechanism, the delayed dissociation rate of these mutant receptors is associated with prolonged survival and even proliferation after removal of the receptor's cytokine ligand of cells expressing the mutant receptors. Additionally, following addition of the cytokine ligand, cells bearing the mutant receptors preferably have decreased receptor tyrosine phosphorylation, ubiquitination and degradation, and slower internalization compared with cells expressing the wild-type receptor.

The mutant RTKIII of the present invention can be any RTKIII now known or later discovered. Preferably, the RTKIII is a CSF-1R, a platelet-derived growth factor receptor (PDGF-R), a stem cell factor receptor (SCFR), or a vascular endothelial growth factor receptor (VEGF-R). More preferably, the RTKIII is a CSF-1R. Where the RTKIII is a CSF-1R, it preferably has a sequence at least 90% identical to SEQ ID NO:1 or SEQ ID NO:2.

The mutant RTKIII of the present invention can be mutated from the RTKIII of any mammal. Preferably, the RTKIII is a human RTKIII; where the RTKIII is a CSF-1R, that CSR-1R is preferably a human CSF-1R. Alternatively, the RTKIII can be a mouse RTKIII, preferably a mouse CSF-1R.

The mutant RTKIIIs of the present invention can be, except for the cysteine-to-serine mutations, wild-type RTKIIIs, or they can further comprise at least one other mutation from the wild-type RTKIII. Non-limiting examples include the R777Q mutant described in Schmid-Antovarchi et al., 1998; the various mutants having mutations at L301 as described in Shurtleff et al., 1990; the tyrosine-to-phenylalanine (Y->F) mutants described in Dey et al., 2000 (see also Yeung and Stanley, 2003 for a discussion of the relevance of these and other tyrosine residues that are subject to phosphorylation); the L301S and Y969F mutants described in Wrobel et al.; the 1562S, Y559F and Y559D mutants described in Rohde et al.; and the Y809F mutant described in Roussel et al., 1990.

Where the mutant RTKIII is a wild-type mouse CSF-1R, it preferably has the sequence of SEQ ID NO:1 except for the C432S and/or C439S mutation. Similarly, where the mutant RTKIII is a wild-type human CSF-1R, it preferably has the sequence of SEQ ID NO:2 except for a C434S and/or C441S mutation.

The invention is also directed to an extracellular domain of any of the mutant RTKIIIs described above. Since the extracellular domain is capable of binding its cytokine ligand, but does not have the transmembrane region or cytoplasmic kinase domain, these invention extracellular domains are useful for binding its cytokine ligand, thus preventing the cytokine from binding a native membrane-bound RTKIII. As such, the extracellular domain could reduce or eliminate activation of the RTKIII by its cytokine ligand.

As used herein, the extracellular domain of SEQ ID NO:1 and SEQ ID NO:2 is amino acid residues 28-439 (i.e., through the conserved cysteines subject to the invention mutations) and 28-441, respectively. The skilled artisan could determine the analogous extracellular domains for any other RTKIII now known or later discovered without undue experimentation.

Preferably, the extracellular domains lack other portions of the mutant RTKIII. Some of these invention extracellular domains further comprise an amino acid sequence that is not part of the mutant RTKIII. These non-RTKIII amino acid sequences, which are preferably fused to the extracellular domain (to form a fusion protein) using genetic methods, e.g., by constructing a vector comprising an in-frame fusion of the extracellular domain with the non-RTKIII amino acid sequence. These non-RTKIII amino acid sequences can serve a variety of purposes, for example facilitating the purification of the extracellular domain, increasing the half-life of the extracellular domain in therapeutic applications, or facilitating dimerization of the receptor extracellular domain (as occurs, for example, when the non-RTKIII amino acid sequence is an immunoglobulin Fc region). Non-limiting examples of useful amino acid sequences here include glutathione-S-transferase, a His-6 tag, a FLAG peptide, or an immunoglobulin Fc region.

Preferred extracellular domains here are from a human CSF-1R with C434S and C441S mutations.

The mutant RTKIIIs described above, and particularly the extracellular domains can be usefully formulated for pharmaceutical applications in a pharmaceutically acceptable carrier.

By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

The above-described mutant RTKIIIs and extracellular domains (including fusion proteins) can be formulated without undue experimentation for administration to a mammal, including humans, as appropriate for the particular application. Additionally, proper dosages of the compositions can be determined without undue experimentation using standard dose-response protocols.

Although the mutant RTKIIIs and extracellular domains can be easily formulated for oral, lingual, sublingual, buccal, intrabuccal, rectal, or nasal administration, it is preferred that they be formulated for parenteral administration, such as for example, by intravenous, intramuscular, intrathecal or subcutaneous injection, since that is the most preferred route of administration of these proteins. Parenteral administration can be accomplished by incorporating the compounds into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

The present invention is also directed to a stable cell line of macrophages lacking a native CSF-1R. Examples of such cells are described in Example 1. These cell lines are particularly useful for evaluating the action of a transgenic CSF-1R that is incorporated into the cells of the cell line.

Preferably, the stable cell line is capable of growing independently of CSF-1. More preferably, the cell line is capable of growing in the presence of a growth factor that is not CSF-1. The growth factor is most preferably granulocyte macrophage-colony stimulating factor, as the cells described in Example 1.

The invention stable cell line can be from any mammalian species. Preferably, the macrophages are mouse macrophages. In other preferred embodiments, the macrophages are human macrophages.

The invention stable cell line described herein can further comprise a transgene. Preferably, the transgene encodes a transgenic protein. More preferably, the stable cell line expresses a transgenic CSF-1R. The transgenic CSF-1R introduced into the stable cell line can be from any species, preferably mouse or human. The transgenic CSF-1R can further comprise at least one mutation from a wild-type CSF-1R. Alternatively, the transgenic CSF-1R is a wild-type CSF-1R. Preferably, the wild-type CSF-1R here is at least 90% identical to SEQ ID NO:1 or SEQ ID NO:2. More preferably, the wild-type CSF-1R here is a mouse CSF-1R having a sequence at least 98% identical to SEQ ID NO:1, or a human CSF-1R having a sequence at least 98% identical to SEQ ID NO:2. Even more preferably, the transgenic CSF-1R is the mutant CSF-1R described above having C432S and/or C439S mutations. Most preferably, this CSF-1R comprises the sequence of SEQ ID NO:1 except for C432S and C439S mutations, or the sequence of SEQ ID NO:2 except for C434S and C441S mutations.

The stable cell line having an active CSF-1R transgene is also preferably capable of giving rise to osteoclasts in the presence of CSF-1 and RANK ligand, as is the capability of macrophages comprising the native CSF-1R.

The invention stable cell line here preferably consists of the MacCsf1r−/− or MacCsf1r+/+ cells described in Example 1, most preferably the MacCsf1r−/− cells. These MacCsf1r−/− cells preferably comprises a transgenic CSF-1R. The transgenic CSF-1R here can be a wild-type CSF-1R having a sequence at least 90% identical to SEQ ID NO:1 or SEQ ID NO:2. Alternatively, the transgenic CSF-1R comprises at least one mutation from a wild-type CSF-1R, preferably the mutant CSF-1R described above having C432S and/or C439S mutations.

Also, the invention is directed to isolated nucleic acids encoding any of the above-described mutant RTKIIIs or extracellular domains. Preparation of any of these nucleic acids is routine to the skilled artisan. Preferably, the isolated nucleic acid is a DNA.

These isolated nucleic acids could comprise the native DNA sequence of the gene or the cDNA, or it could have any amount of substitutions from the native sequence, e.g., to utilize more preferred codon usage, or for any other reason.

The invention is additionally directed to a vector comprising the above isolated nucleic acid and additional genetic elements allowing transformation, expression and secretion of the mutant RTKIII or extracellular domain in a mammalian cell. These compositions are not limited to any particular vectors and could encompass a naked DNA vector such as a plasmid, a viral vector, or any other suitable vector type now known or later discovered. Preferably, the mammalian cell in which the vector is capable of transformation, expression and secretion is a macrophage or osteoclast.

The inventors have also developed improved methods of preparing certain stable cell lines. See Example 1. Thus, the invention is further directed to a method of preparing a stable cell line of macrophages from a mammal where the stable cell line maintains CSF-1 responsiveness. The method comprises: isolate macrophages from the mammal; culture the macrophages in growth medium comprising CSF-1; immortalize the macrophages using an SV-U19-5 retrovirus; single cell plate the macrophages to create a cloned cell line; and culture the cloned cell line to create the stable cell line. The SV-U19-5 retrovirus is described in Jat and Sharp, 1986. Other immortalized macrophage cell lines are described in Gandino and Varesio, 1990; Morgan et al., 2005; Roberson and Walker, 1988; Stabel and Stabel, 1995; Wilson et al., 1991; Pirami et al., 1991, and Pollard et al., 1991. A stable cell line made by these methods is also envisioned.

Because immortalized macrophage cell lines can be heterogeneous (see Pirami et al., 1991, and Pollard et al., 1991), it is preferred that the cloned cell lines be tested for CSF-1 responsiveness, to be sure that characteristic of the line is not lost.

The macrophages for these invention methods can be from any mammal. Preferably, the macrophages are from a mouse or a human, most preferably from a mouse.

The macrophages can be from any source in the mammal, e.g., bone marrow, liver (including fetal liver), peritoneum, thymus, spleen or brain. Preferably, the macrophages are from bone marrow.

The invention is additionally directed to a method of preparing the invention stable cell line described above lacking a native CSF-1R. The method comprises: isolate macrophages from a Csf1r-/Csf1r- mammal; culture the macrophages in growth medium comprising granulocyte-macrophage colony stimulating factor; immortalize the macrophages; single cell plate the macrophages to create a cloned cell line; and culture the cloned cell line to create the stable cell line. The macrophages can be immortalized by any method now known or later discovered. Preferably, the macrophages are immortalized with a retrovirus. The most preferred retrovirus is an SV-U19-5 retrovirus.

The macrophages for these invention methods can be from any mammal. Preferably, the macrophages are from a mouse or a human, most preferably from a mouse.

The macrophages can be from any source in the mammal, e.g., bone marrow, liver (including fetal liver), peritoneum, thymus, spleen or brain. Preferably, the macrophages are from bone marrow.

In the most preferred aspects of these methods, the macrophages are from mouse bone marrow and the retrovirus is an SV-U19-5 retrovirus.

The invention is additionally directed to a method of treating a mammal having or at risk for undesirable activation of a native RTKIII. The method comprises administering to the mammal the above described extracellular domain, where the extracellular domain is from the same type of RTKIII as the native RTKIII. These methods are particularly useful where the mammal has a disease or disorder where activation of the native RTKIII is involved. Such diseases or disorders are discussed in, for example, Yeung and Stanley, 2003; Stanley, 2000; Chitu and Stanley, 2006; Aharinejad et al., 2002; Aharinejad et al., 2004; Paulus et al., 2006; Sapi, 2004; Pixley and Stanley, 2004; Floege et al., 2003; Lotinun et al., 2003; Lennartsson et al., 2005; and Dai et al., 2002.

In one aspect of these invention methods, the extracellular domain protein is administered to the mammal, e.g., by intravenous administration. In another aspect, the extracellular domain is administered by administering a vector encoding the extracellular domain to the mammal such that the extracellular domain is expressed in the mammal.

The mutant RTKIII for these invention methods can be any RTKIII now known or later discovered. Preferably, the RTKIII is a CSF-1R, a platelet-derived growth factor receptor (PDGF-R), a stem cell factor receptor (SCFR), or a vascular endothelial growth factor receptor (VEGF-R). More preferably, the RTKIII is a CSF-1R. The extracellular domain is preferably of a human CSF-1R having an amino acid sequence at least 99% homologous to SEQ ID NO:2 with C434S and C441S mutations.

Where the extracellular domain is from a CSF-1R, it is envisioned that these invention methods are particularly useful when the mammal is a human that has cancer or an inflammatory disease exacerbated by CSF-1. The methods are expected to be even more useful when the human has a cancer tumor at risk for metastasis. Alternatively, the human can have an autoimmune disorder and/or arthritis. Preferred autoimmune disorders are lupus, rheumatoid arthritis and osteoarthritis. The methods are also particularly useful when the mammal is a human with an allograft or xenograft, or when the mammal is a human that has HIV-1 encephalitis, Alzheimer's disease, Langerhans cell histiocytosis, a brain tumor or a brain injury. In other aspects of these methods, the mammal is a human that has atherosclerosis and/or obesity.

The methods are also particularly useful when the mammal is a human with a disease or disorder involving inflammation. Non-limiting examples of such diseases or disorders are proliferative vascular disease, acute respiratory distress syndrome, cytokine-mediated toxicity, psoriasis, interleukin-2 toxicity, appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, hepatitis, inflammatory bowel disease, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcets's syndrome, allograft rejection, graft-versus-host disease, ankylosing spondylitis, Berger's disease, type 1 diabetes, type 2 diabetes, Berger's disease, Retier's syndrome, and Hodgkins disease.

In other aspects of these invention methods, the mutant and native RTKIII is a PDGF-R having an amino acid sequence at least 80% identical to SEQ ID NO:4. Preferably, the mammal is a mouse or a human. These methods are particularly useful when the mammal has a renal disease, chronic hyperparathyroidism, cancer, rheumatoid arthritis or myocarditis.

Alternatively, the mutant and native RTKIII can be a SCFR having an amino acid sequence at least 80% identical to SEQ ID NO:3. Preferably, the mammal is a mouse or a human. These methods are particularly useful when the mammal has a cancer involving excess SCFR, or mastocytosis.

The mutant and native RTKIII can also be a VEGF-R having an amino acid sequence at least 80% identical to SEQ ID NO:5. Preferably, the mammal is a mouse or a human. These methods are particularly useful when the mammal has wet age-related macular degeneration, retinopathy of prematurity, a cancer involving excess VEGF, or an inflammatory disease involving VEGF.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1

### Role of CSF-1R Y559 and Y807 in Macrophage Proliferation and Differentiation Signaling

### Example Summary

Colony stimulating factor-1 (CSF-1) is the major regulator of development and maintenance of tissue macrophages and also primes and regulates other responses in macrophages. Analysis of CSF-1R structure/function relationships in macrophages is therefore of critical importance to understanding the role of CSF-1 and macrophages in development and disease. To facilitate these studies, a CSF-1R-deficient MacCsf1r−/− macrophage cell line was developed that can be maintained in GM-CSF. Retroviral expression of the wild type (WT) CSF-1R in MacCsf1r−/− cells rescues the CSF-1-induced survival, proliferation, differentiation and morphological characteristics of primary macrophages. Retroviral transduction of mutated CSF-1Rs into MacCsf1r−/− cells allows dissection of CSF-1R function in the macrophage. Individual expression of 8 single intracellular domain Y->F mutations (544, 559, 697, 706, 721, 807, 921, 974) rescued the CSF-1-inducible phenotypes to varying degrees, whereas expression of CSF-1R YEF, in which all 8 Ys were mutated to F, failed to rescue. The activation loop Y807F mutation severely compromised proliferation, differentiation and survival and the juxtamembrane domain Y559F mutation reduced proliferation and differentiation. Both Y559 and Y807 “add-back” (AB) receptors in which these Ys were individually present on the YEF background significantly rescued the proliferation response and the Y559,807 double AB restored the requirement for CSF-1 and CSF-1 stimulated survival and proliferation. These studies demonstrate that the MacCsf1r−/− macrophage line is an suitable platform in which to perform CSF-1 structure/function studies and that CSF-1R juxtamembrane and activation loop tyrosines, Y559 and Y807, as shown in other systems, play important roles in regulating macrophage survival and proliferation.

### Materials and Methods.

Reagents. Anti-pY100, anti-CSF-1R, anti-ubiquitination, anti-CSF-1R(pT809), anti-Shc, and anti-Grb2 was from Transduction Laboratories.

Site-directed mutagenesis and retroviral constructs. The retroviral vector pMSCV-IRES-GFP (Persons et al., 1999) and the ecotropic, replication-defective helper virus pSV-E-MLV (Muller et al., 1991) cDNAs were gifts of Drs. A. W. Nienhuis (St. Jude Children's Research Hospital, Memphis, Tenn.) and O. N. Witte (University of California Los Angeles, Los Angeles, Calif.), respectively. The SV-U19-5 retrovirus containing a variant of the SV40 Large T antigen (Jat and Sharp, 1986) was a gift from Dr. P. S. Jat. A pGEM-2 plasmid containing an EcoRI fragment including the complete c-fins c-DNA (nucleotides 1-36656, accession number NM_{—}007779) and the pZen113xNc-FMS YQF/Y559F/Y974F plasmid in which six tyrosines are mutated to phenylalanine, were gifts from Dr. L. R. Rohrschneider. Site-directed mutagenesis was performed using a kit according to the manufacturer's instructions (Stratagene, cat# 200518). All introduced mutations were confirmed by sequencing. The coding regions of mouse wild type (WT) or mutant CSF-1R cDNAs were inserted into the MSCV-IRES-GFP vector at the EcoRI or EcoRI/XhoI site upstream of the IRES driving expression of GFP.

Derivation of the cloned MacCsf1r−/− and MacCsf1r+/+cell lines. Bone marrow-derived macrophages (BMM) from Csf-1r−/− and Csf-1r+/+ outbred mice (Dai et al., 2002) were prepared as described in Stanley (1998) with the following modifications. Cells were cultured in BMM medium (α-MEM supplemented with 3.4 μl/liter β-mercaptoethanol, 0.29 g/liter glutamine and 0.2 g/liter asparagine, containing 15% fetal calf serum (FCS)) containing IL-3 and 2% GM-CSF conditioned medium (GMCM) (rather than IL-3 and CSF-1) and the adherent cells were harvested between days 5 and 8 of culture (rather than between days 3 and 6). Both Csf-1r−/− BMM and Csf-1r+/+ BMM were immortalized by infection with the SV-U19-5 retrovirus (Jat and Sharp, 1986). Briefly, the medium in 100 mm dishes of sub-confluent BMM was replaced with diluted SV-U19-5 viral supernatant in BMM medium containing 8 μg/ml polybrene and 2% GMCM and incubated overnight. The cells were then washed once with phosphate buffered saline, the medium replaced with BMM medium containing GM-CSF, the cells cultured until almost confluent and then split 1:5 in the same medium containing 250 μg/ml G418 with medium changes every 4 days for approximately 10 days. Independently arising clones of transformed MacCsf1r−/− (M−/−) and MacCsf1r+/+(M+/+) macrophages were purified by single cell plating and culture in 96-well microplates (Falcon, 353072), in Dulbecco's modified minimal essential medium (Invitrogen, Carlsbad, Calif.) containing 10% newborn calf serum (NCS) (Invitrogen) (Macrophage medium), 2% GMCM and 50 μg/ml G418.

Retroviral transfection of MacCsf1r−/− cells. For MSCV retroviral infection, human kidney 293T cells, cultured in 100 mm culture dishes with Dulbecco's modified Eagle's medium (DMEM) containing 10% FCS were transfected with the pMSCV-IRES-GFP (12 μg) and PSV-Ψ-E-MLV (12 μg) DNAs using calcium phosphate precipitation. The medium was changed to fresh 293T culture medium 24 h post-transfection. At 48 h-post-transfection, the retroviral supernatant was harvested and filtered through a 0.45-μm filter. Sub-confluent cultures of MacCsf1r^{-/-} cells in 100 mm plates were incubated with the fresh retroviral supernatants in the presence of 2% GMCM and 4 μg/ml polybrene for 24 h, prior to replacing the medium with fresh 2% GMCM medium and culturing the cells for a further 4-7 days. Cultured cells were harvested by cell scraping and subjected to fluorescence-activated cell sorting (FACS) for GFP⁺ cells. Sorted cells (25-70% GFP⁺) were expanded by further culture in 2% GMCM medium and subjected to Western blot analysis for CSF-1 R expression. Generally, GFP expression correlated with CSF-1R expression. If fewer than 90% of the expanded cells were GFP⁺ or if the level of CSF-1R expression was higher or lower than the level of expression in M+/+ cells, cell populations were resorted for GFP⁺ cells into high, medium and low GFP expressers and the sub-populations tested to choose lines with CSF-1R expression levels approximating those of MacCsf1r+/+cells. Further selections were made on the basis of cell surface CSF-1R expression (see below). Cells cultured in GM-CSF or CSF-1 for 3 months maintained stable CSF-1R expression and cells thawed for experiments were passaged for no longer than 2 months.

FACS and FACScan analysis. Retrovirally infected cells were sorted for GFP+ cells using a FACSVantage SE cell sorter (BD Biosciences, San Jose, Calif.). Cell surface expression of the CSF-1R was determined by FACScan analysis using the monoclonal anti-CSF-1R AFS98 antibody (gift of Dr. S. Nishikawa) (Sudo et al., 1995). Mac1 expression was determined using a R-phycoerythrin (R-PE)-conjugated rat anti-mouse CD 11b (integrin-M chain, Mac-1-chain) monoclonal antibody (BD Pharmingen™, cat #557397).

CSF-1 stimulation, western blot and immunoprecipitation. Subconfluent 100 mm dish cultures of cells were starved of growth factor for 16 h to upregulate CSF-1 receptor expression, then incubated with 360 ng/ml purified human recombinant CSF-1 at 37° C. or 4° C. Cells were solubilized in lysis buffer (1% NP-40, 10 mM Tris-HCl, 50 mM NaCl, 30 mM Na₄P₂O₇, 50 mM NaF, 100 μM Na₃VO₄, 5 μM ZnCl₂, 1 mM benzamidine, 10 μg/ml leupeptin and 10 μg/ml aprotinin, pH 7.2) or SDS sample buffer as described previously (Berg, 1999). For the detection of CSF-1R expression by Western blotting, 50 μg of each SDS cell lysate was separated by 7% SDS-PAGE and transferred to a polyvinylidene dufluoride (PVDF) membrane. Equal protein loading was confirmed by blotting with anti-actin antibody. For immunoprecipitation, lysates were incubated with anti-CSF-1R antibodies and protein G-Sepharose beads (Zymed) overnight at 4° C. The beads were washed five times with wash buffer at 4° C. prior to elution of the bound proteins with SDS sample buffer at 65° C. for 10 min. Protein determinations, gradient (7.5-17.5% acrylamide) SDS-PAGE and Western blots were performed as described previously (Yeung, 1998).

In vitro receptor autophosphorylation assay. Cells were starved for 16 h, then stimulated with CSF-1 (4° C., 2 h). Cells were then lysed and the lysates subjected to immunoprecipitation with either 5 μg of either anti-CSF-1R antibody or unrelated IgG as a control. The immunoprecipitates were washed and incubated at 4° C. for 20 min in 5 mM ATP dissolved in kinase assay buffer (50 mM HEPES, pH7.3, 15 mM MnCl₂, 8 mM MgCl₂, 0.2% NP40, 10 μg/ml leupeptin, 10 μg/ml aprotinin). The beads were then washed again and the CSF-1R immunoprecipitates eluted with SDS sample buffer at 65° C. for 10 min, subjected to SDS-PAGE (%) electrophoresis, transfer to PVDF membrane and Western blotting for phosphotyrosine and the CSF-1R.

Cell Proliferation assays. Two methods for cell proliferation assay were used. The DAPI DNA staining method was modified from the Quantos™ cell proliferation assay kit (Stratagene cat# 302011). Cells previously cultured in GMCM medium were seeded at 5×10³ cells per well in macrophage medium containing 36 ng/ml of human recombinant CSF-1 (a gift from Chiron Corp) in a 48 well plate (Corning Costar 48-well Cat# 3548) with at least 3 wells per data point. The medium was changed every 3 days.

For each day of the growth curve, one plate was cooled to −80° C. for 15 min, the contents thawed at room temperature and 200 μl of 1 μg/μl DAPI in staining buffer (100 mM Tris, pH 7.4, 150 mM CaCl₂, 0.5 mM MgCl₂, 0.1% Nonidet-P-40) added to each well, prior to incubation of the plate (1 h, 20° C.) and reading of the fluorescence in wells using a microplate reading fluorometer with filters appropriate for 355-nm excitation and 460-nm emission. Data are expressed as the average fluorescence after subtraction of the average blank (no cell) value. Alternatively, cells were plated at 2×10⁴ cells in 35 mm well dishes in 2 ml of macrophage medium with or without 120 ng/ml CSF-1 and cultured with medium changes every 2 days. Cell counts were performed daily on triplicate cultures. Adherent macrophage nuclei were recovered using 0.005% Zwittergen, diluted in Isoton II (Curtin Matheson Scientific, Inc.), and the nuclei counted using a Coulter Counter ZM.

Macrophage differentiation assay. For cell surface Mac1 expression assays, cells were cultured in 2% GMCM to semi-confluence, prior to incubation in 36 ng/ml CSF-1 for a further 3 days. The cells were harvested, washed once with phosphate buffered saline (PBS) and incubated with R-PE conjugated Mac1 antibody or a control, unrelated antibody in assay buffer (1% bovine serum albumin (BSA) in PBS) at 4° C. for 20 min. Cells were washed twice with assay buffer and subjected to FACS analysis. The relative expression level of Mac1 was determined as the difference between the geometric means of fluorescence densities for Mac1 and control the monoclonal antibody expressed as a percentage of the difference for cells expressing the WT CSF-1R.

### Results

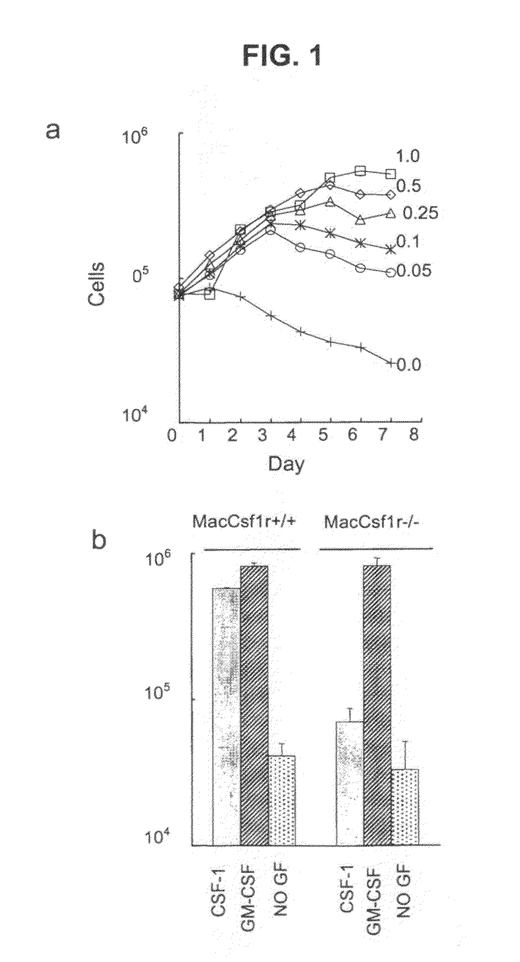

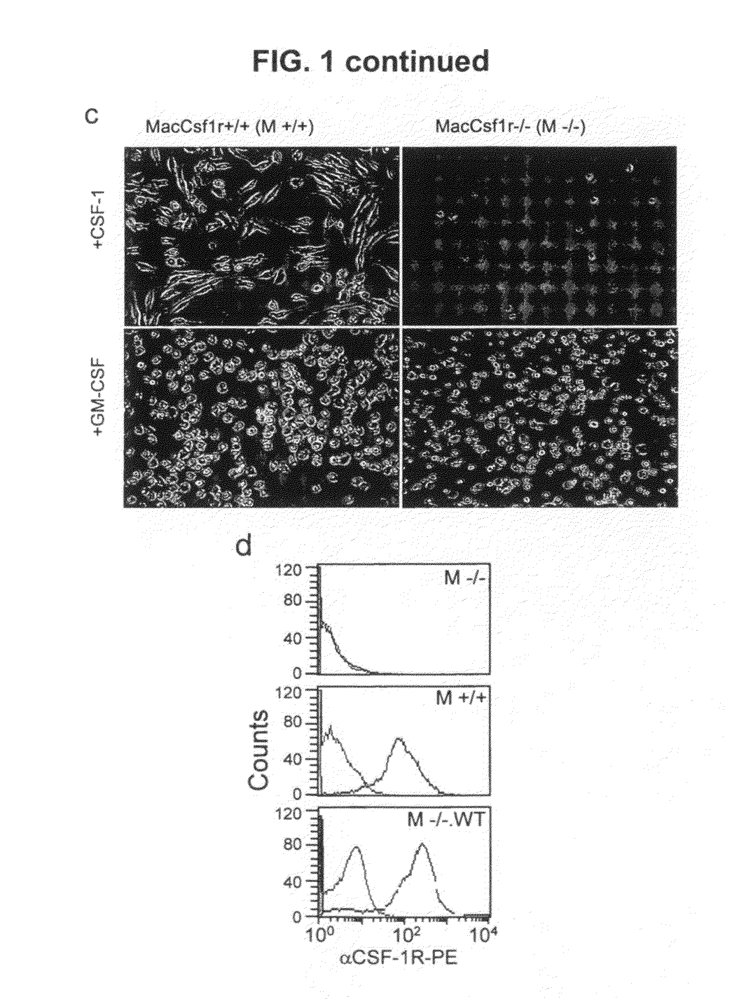

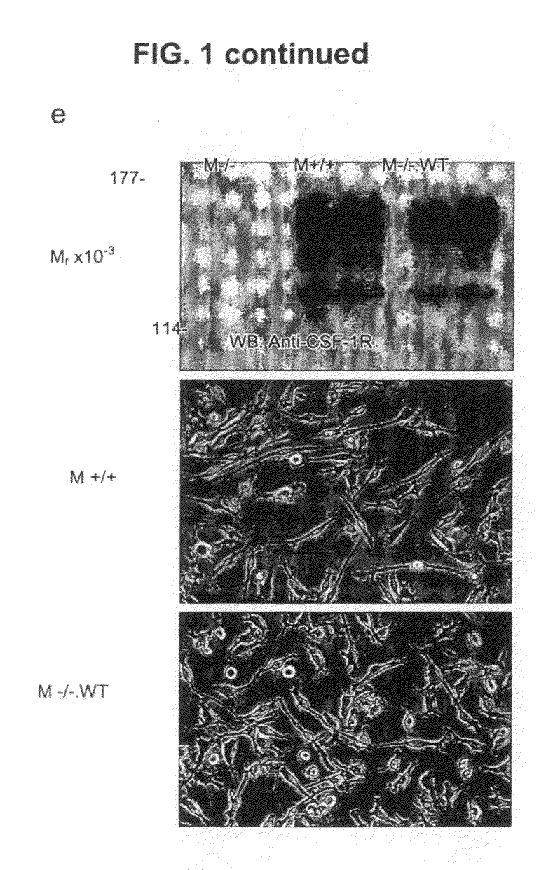

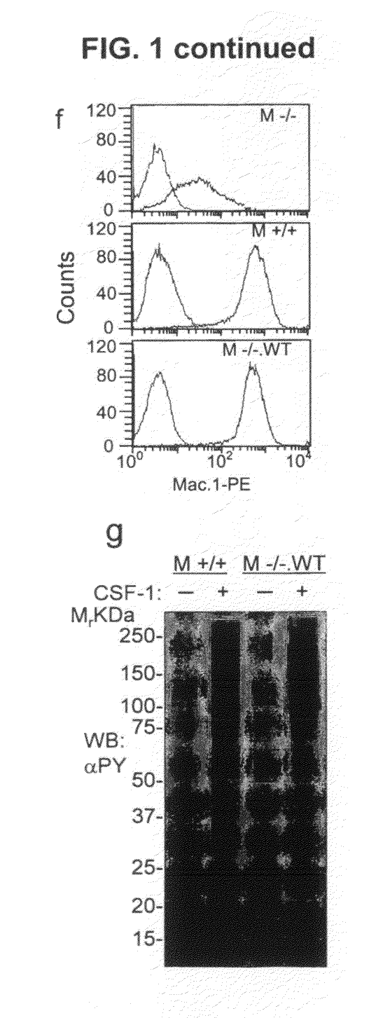

Characteristics of macrophages of the MacCsf1r+/+ (M+/+) and MacCsf1r −/− (M −/−) mouse macrophage cell lines. M−/− and M+/+ cells exhibited normal a normal proliferative response to GM-CSF and die in the absence of growth factor (FIGS. 1a, b). In contrast to M+/+ cells, which exhibit a normal macrophage proliferative response, M−/− cells die in the presence of CSF-1 (FIGS. 1a, b). The morphology of M+/+ and M−/− cells cultured in GM-CSF is similar and they are much rounder than the elongated M+/+ cells grown in the presence of CSF-1 (FIG. 1c). Retroviral expression of the wild type (WT) CSF-1R in M−/− cells at levels normally found on BMM and in M+/+ cells (FIGS. 1d, e), confers a proliferative response to CSF-1 and elongated mature cellular morphology in the presence of CSF-1 that mimics the behavior of M+/+ cells (FIG. 1e) and BMM (data not shown). These M−/−.CSF-1R_WT (M−/−.WT) cells grown in GM-CSF, when transferred to CSF-1 for 3 days, exhibited a dramatic increase in the expression of the Mac1 macrophage differentiation marker, up to the level of its expression in M+/+macrophages (FIG. 1f). In addition, M−/−.WT cells exhibited a CSF-1R tyrosine phosphorylation response that is indistinguishable from the response of M+/+ cells (FIG. 1g). These results demonstrate that M−/− cells are appropriate for structure-function studies of the CSF-1R in the regulation of macrophage survival, proliferation and differentiation.

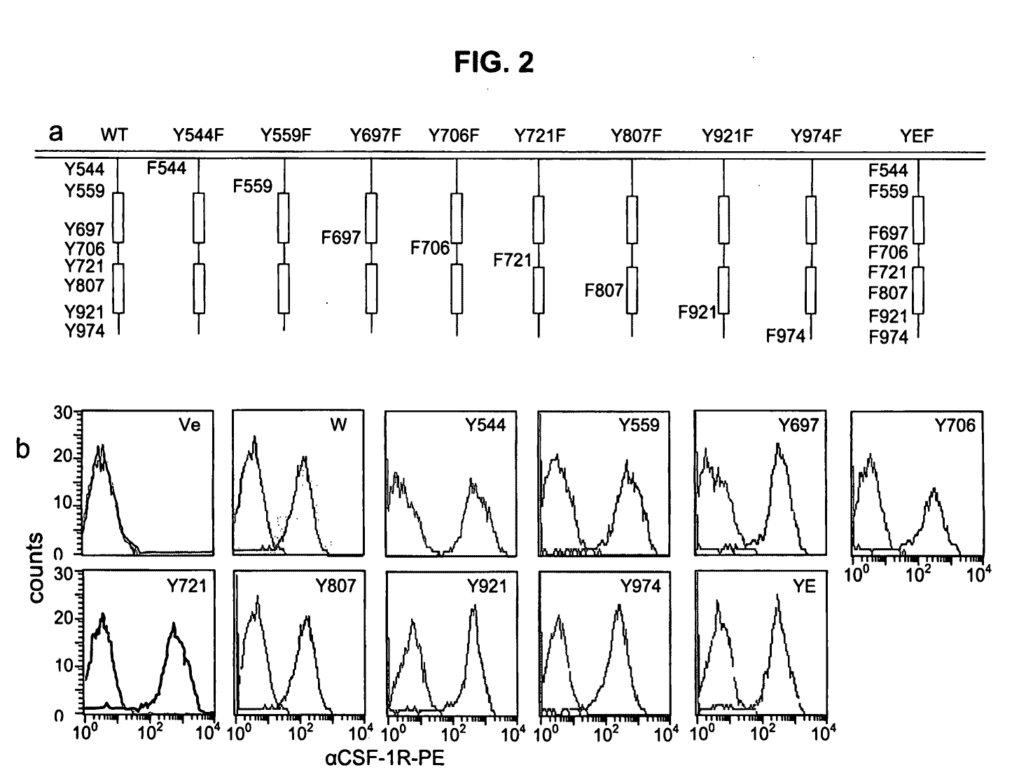

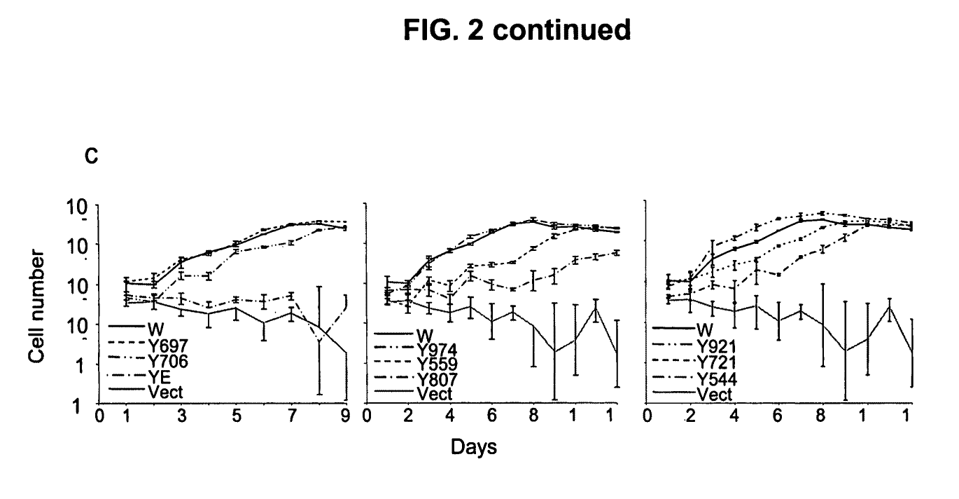

Role of individual tyrosines in CSF-1R-regulated macrophage proliferation. In studies with the WT CSF-1R expressed in hematopoietic cells or fibroblasts, 7 intracellular domain tyrosines are known to be phosphorylated in the response to CSF-1. In addition, Y544 is tyrosine phosphorylated in the oncogenic form of the CSF-1R protein encoded by v-fms. To examine the roles of individual CSF-1R tyrosines in CSF-1 regulated macrophage survival, proliferation and differentiation, 8 M−/− cell lines were created expressing CSF-1Rs bearing unique Y->F mutations and one (M−/−.CSF-1R_YeightF (M−/−.YEF)) in which all 8 tyrosines were mutated to phenylalanine (FIG. 2a). Following upregulation after culture in GMCM, each of these cell lines expressed wild-type levels of the CSF-1R on SDS-PAGE and Western blotting of whole cell lysates (data not shown) and wild type levels of cell surface CSF-1R (FIG. 2b). The proliferation characteristics of cells of all 8 lines, together with vector infected M−/− cells (M−/−.vec) and M−/−.wtCSF-1R were examined simultaneously (FIG. 2c). The growth curve for M−/−.YEF_CSF-1R cells was virtually indistinguishable from the curve for M−/−.vec cells (FIG. 2c). The most severe effects on proliferation rate were observed with the activation loop mutation Y807F and the juxtamembrane domain mutation Y559F (FIG. 2c). The doubling times of the Y807F and Y559F mutations were increased. The two interkinase domain mutation lines M−/−.Y706FCSF-1R and M−/−.Y721FCSF-1R exhibited a slight reduction in proliferation rate compared with the M−/−.wtCSF-1R line (FIG. 2c). The doubling time for the M−/−.Y544FCSF-1R line was closer to that of the M−/−.wtCSF-1R line (data not shown). The proliferation rates of M−/−.Y697FCSF-1R and M−/−.Y974FCSF-1R lines were indistinguishable from those of M−/−.wtCSF-1R cells, whereas M−/−.Y921F_CSF-1R cells had a higher proliferation rate.

Role of individual tyrosines in CSF-1R-regulated macrophage survival. Since changes in cell number result from a balance between cell survival and cell death, we determined the effect of the Y->F mutations on survival by incubating cells of each of the lines for 4 days with daily medium changes in 600 pg/ml CSF-1 at a concentration that induces survival of wild type macrophages without significant proliferation (Tushinski et al '82). Compared with M−/−.YEF and M−/−.vec cells cultured in 600 pg/ml CSF-1 and M−/−.WT cells cultured in the absence of CSF-1, in which there was a loss in viable cell numbers over 4 days of culture, cells of each of the single Y->F mutant CSF-1R expressing lines survived. There was substantial proliferation of M−/−.WT cells grown under the same conditions in the presence of 120 ng/ml CSF-1. A significantly increased proliferation was also observed with M−/−.Y974F cells grown in 600 pg/ml CSF-1, consistent with negative regulation of proliferation by this tyrosine. Compared with the other single Y mutant cell lines, M−/−.Y807F cells had not increased in number by 4 days and could not be maintained in 120 ng/ml CSF-1 (data not shown, indicating that Y807 was required for CSF-1-induced cell survival). These results indicate that Y807 plays a significant role in regulating CSF-1-induced macrophage survival and that individual mutations of the other tyrosines are without effect.

Role of individual tyrosines in CSF-1R-regulated macrophage morphology. Compared with their morphology when cultured in GM-CSF, the morphology of BMM grown in the presence of CSF-1 is significantly more elongated and they are more adherent, phenotypes preserved in M+/+ and M−/−.wtCSF-1R macrophages (FIG. 1c). To examine of the morphology of macrophages in M−/− cell lines expressing CSF-1Rs bearing unique Y->F mutations, the cells were cultured to ˜50% confluence in GM-CM, the medium replaced with CSF-1 medium and the cells cultured for a further 1-2 weeks in the presence of CSF-1. Experiments with M−/−.wtCSF-1R macrophages revealed that at least 1 week in culture with CSF-1 was required for the cells to fully transform from the GM-CSF morphology to the CSF-1 morphology. Thus these studies could not be carried out with M −/−.YEF_CSF-1R macrophages, which did not survive in CSF-1. However, the variation in phase contrast morphology when cultured in CSF-1, between the macrophage lines expressing different Y->F point mutations in the CSF-1R, indicate that the phosphorylation of individual tyrosines control important aspects of macrophage morphology. In particular, M−/−.Y559F_CSF-1R and M−/−.Y974F_CSF-1R macrophages exhibited much decreased spreading and M−/−.Y721F_CSF-1R and M−/−.Y974F_CSF-1R macrophages a loss of elongated morphology, whereas the phenylalanine mutations in the remaining tyrosines, Y544, Y697, Y706 and Y921, had little effect on morphology under phase contrast. These results demonstrate that specific Y->F CSF-1R mutations affect CSF-1-stimulated macrophage spreading and/or elongated morphology.

Role of individual tyrosines in CSF-1R-regulated macrophage differentiation. The Y->F CSF-1R mutant cell lines were also examined for their capacity to express the macrophage differentiation marker, Mac1, following a 3-day incubation with CSF-1 as shown for M−/−.wtCSF-1R cells in FIG. 1f. M−/−.VEC cells exhibited only 13% of the Mac1 expression of M−/−.wtCSF-1R cells. Consistent with the retention of some morphological character, M−/−.YEF cells cultured with CSF-1 expressed 21% of Mac1 expression of M −/−.wtCSF-1R cells. Among the other Y->F mutants, the Y807F and Y559F mutations lowered CSF-1-stimulated Mac1 expression substantially (23% and 30% respectively). Other mutations with major effects were Y921F (48%) and the Y706F, which resulted in a substantial increase in CSF-1-induced expression of Mac1 (154%), correlating with its elongated morphology. The Y544F, Y697F, Y721F and Y974F mutations only slightly suppressed the differentiation response (˜67%). These results indicate that specific Y->F CSF-1R mutations can positively and negatively affect CSF-1-stimulated macrophage differentiation. Overall, the broad variation of the morphologies and Mac1 expression levels among the cells expressing individual CSF-1R Y->F point mutations, indicate that these individual tyrosines control important aspects of morphology and differentiation of macrophages. Characteristics of M−/−.CSF-1R Y559AB and M−/−.CSF-1R Y807AB macrophages. Phosphorylation of the juxtamembrane domain Y559 is believed to be important for the relief of autoinhibition in class III receptor tyrosine kinases and phosphorylation of the activation loop Y807 is believed to be critical for kinase activation. Consistent with these observations, the Y807F mutation significantly affected CSF-1-regulated proliferation, survival and differentiation and the Y559F mutation had major effects on CSF-1-regulated proliferation and differentiation. To further understand the function of tyrosines 559 and 807 M−/−.CSF-1R_Y559AB and M−/−.CSF-1R_Y807AB cell lines were created in which all 8 tyrosines with the exception of Y559 and Y807 were mutated to phenylalanine. M−/−.CSF-1R Y559AB and M−/−.CSF-1R Y807AB cell lines expressed cell surface levels of the CSF-1R approximating those of the other cell lines. Cells of both lines possessed an approximately equal ability to proliferate in the presence of CSF-1 at rates approaching those of M−/−.wtCSF-1R cells although their ability to differentiate was weak (data not shown). A M−/−.CSF-1R Y559,807AB cell line was then created in which all 8 tyrosines with the exception of Y559 and Y807 were mutated to phenylalanine. The Y559,807 double AB restored the requirement for CSF-1 and CSF-1 stimulated survival and proliferation.

### Discussion

A novel CSF-1R-deficient macrophage line is described herein that can be used to examine the structure-function relationships of the CSF-1R in context of the macrophage, the predominant CSF-1-regulated cell type in which the regulation of survival, proliferation, differentiation and function are controlled by the CSF-1R. This system, compared with others involving either CSF-1R-transfected fibroblasts or myeloid cells or erythropoietin receptor-CSF-1R hybrids in bone marrow precursor cells allows structure-function analysis of the full-length receptor in the correct cellular context with sufficient cell numbers for the use of proteomic approaches. The M−/− macrophage cell line allows analysis of CSF-1R-regulated pathways regulating survival, proliferation, differentiation, morphology, motility and function. Furthermore, as bone marrow derived macrophages can be stimulated to differentiate to osteoclasts, it is likely that M−/−.CSF-1R_WT cells cultured with CSF-1 and RANK ligand this line will differentiate to osteoclasts and that the M−/− line can therefore also be used to study the role of the CSF-1R in regulating osteoclast differentiation and function.

A preliminary survey of individual mutations of tyrosines known to be phosphorylated in the response of the CSF-1R to CSF-1, or in the activated v-fins oncoprotein, indicates that individual mutations differentially contribute to these responses. However, two tyrosine phosphorylation sites, the juxtamembrane Y559 and activation loop Y807, are important for the proliferative response in that Y->F mutations at these sites reduce the proliferative responses considerably and Y add-back mutations at these sites restore significant proliferative responses to the receptors devoid of the other Y phosphorylation sites. Interestingly, despite the importance of Y559 and Y807 for differentiation their combined add back did not restore the ability to express high levels of Mac1, suggesting that additional receptor Ys can contribute significantly to the differentiation response. Both Y559 and Y807 are believed to be important for CSF-1R activation, Y559 phosphorylation for the relief of negative autoinhibition and Y807 for receptor kinase activity. The demonstration that they are sufficient for restoration of CSF-1-induced macrophage proliferation is of interest in the context of the demonstration that several of the other tyrosines are important for CSF-1-induced morphological changes (Y706, Y721 and Y974) and differentiation (Y706 and Y929). These Ys, not required for the proliferative response, are therefore likely to be involved in the regulation of other aspects of the macrophage response to CSF-1, including chemotaxis and differentiation.

### Example 2

### Extracellular Domain Mutations that Confer Increased Affinity of the CSF-1 Receptor for CSF-1

Example 1 discloses an immortalized mouse macrophage cell line (M+/+) that requires the macrophage growth factor CSF-1 for survival, proliferation and maintenance of the fully differentiated macrophage phenotype. A mouse Mac.Csf1r−/− macrophage cell line (M−/−) was also developed that lacks the CSF-1 receptor (CSF-1R). Retroviral transduction of the mouse CSF-1R into M−/− cells creates an M−/−.WTCSF-1R line, the cells of which survive, proliferate and maintain the fully differentiated macrophage phenotype like M+/+ cells. M−/− cells that do not express the CSF-1R can be grown in granulocyte macrophage-colony stimulating factor (GM-CSF), which acts independently of the CSF-1R. The effects of mutations in the CSF-1R were examined by retrovirally transducing the mutant receptors into M−/− cells. M−/− cells expressing mutant receptors are maintained by culture in GM-CSF. Using this system, the properties of cells expressing 2 point mutations in the CSF-1R extracellular domain that increase the affinity of the CSF-1R for CSF-1 by decreasing the CSF-1 dissociation rate were studied. The results leading to these findings are summarized below.

Retroviral vectors as described in Example 1 were made comprising DNA encoding mutant mouse CSF-1Rs. The mutant CSF-1Rs had one or two substitutions at the invariant cysteines at positions 432 and 439. Six vectors were synthesized. Two had substitutions at 432, one substituted with serine (C432S) and the other substituted with alanine (C432A); two had analogous substitutions at 439 (C439S and C439A); and the remaining two had analogous substitutions at both 432 and 439 (C432S/C439S). The six vectors were each introduced into separate M−/− cell cultures, as was a vector with wild-type mouse CSF-1R.

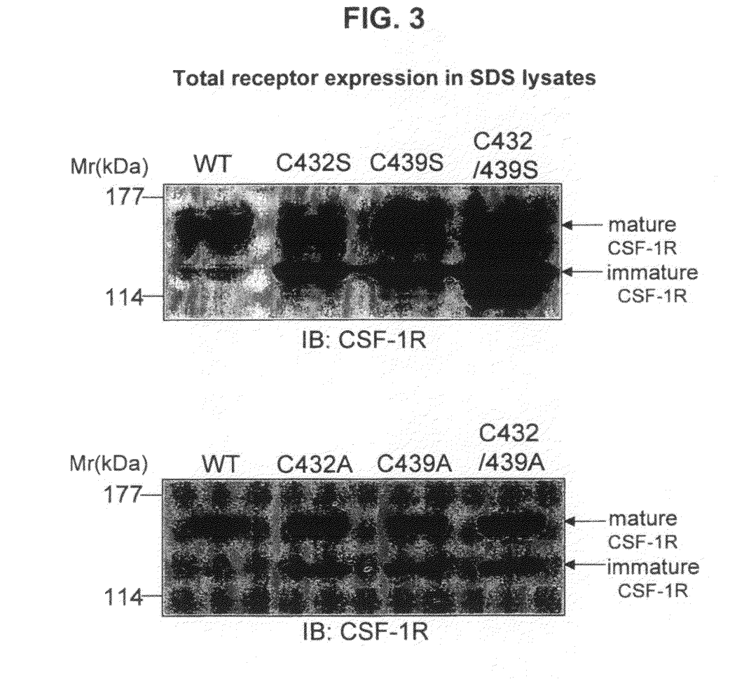

Total CSF-1R expression in SDS lysates was measured in M−/− cells expressing the WT CSF-1R and various extracellular domain (ECD) mutant CSF-1Rs. As shown in FIG. 3, the M−/− cells expressing the wild type (M−/−.WTCSF-1R) and mutant receptors expressed approximately equivalent amounts of mature CSF-1R. However, the macrophages expressing the mutant CSF-1Rs expressed more immature CSF-1R than the macrophages expressing the WT CSF-1R. This is likely to be due to a disruptive effect of the mutations on CSF-1R protein folding in the endoplasmic reticulum and the subsequent accumulation of immature mutant CSF-1Rs in this location. This effect was more pronounced for the C->S mutants than for the cells transduced with the CSF-1R having the alanine substitutions (M−/−.C432ACSF-1R, M−/−.C439ACSF-1R, and C432A, C439A, and C432A/C439A). The cells transduced with these C->A mutant CSF-1Rs are control cells for mutation of cysteines 432 and 439 to serine and were further studied (see below).

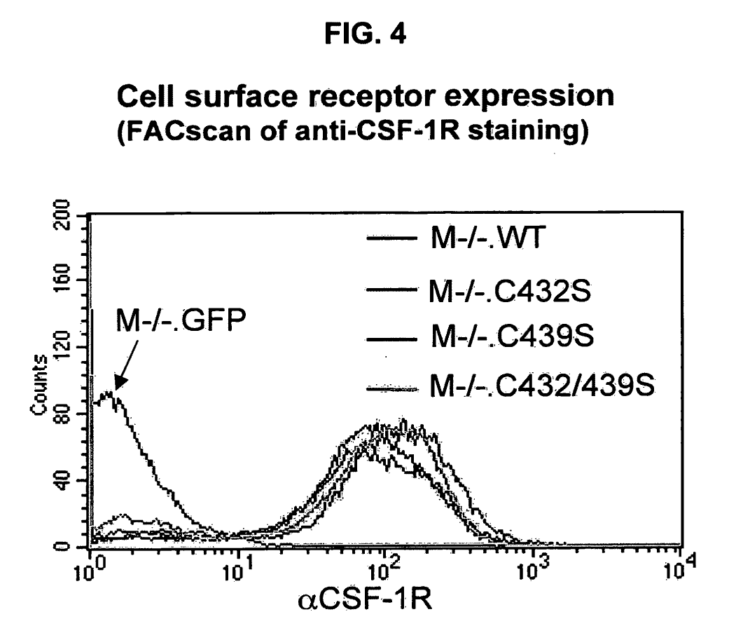

M−/− cells comprising the CSF-1R C-S mutations and M−/− cells WT CSF-1R were stained with anti-CSF-1R and analyzed by FACS. FIG. 4 shows the results. There was equivalent expression by FACS of the cell surface CSF-1Rs on M−/− cells expressing the WT CSF-1R and the mutant CSF-1Rs. A similar result was obtained for the M−/− cells expressing the corresponding C->A mutations (data not shown).

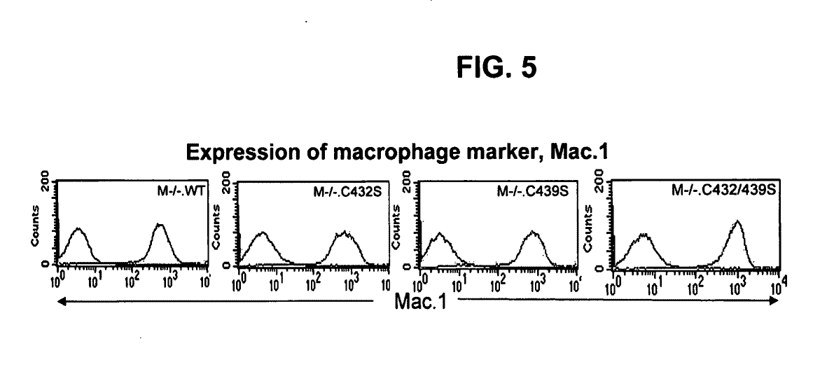

Differentiation in these cells were analyzed by FACS after staining for Mac.1. As shown in FIG. 5, there was equivalent differentiation (as measured my Mac1 positivity) of M−/− cells expressing the WT CSF-1R and the mutant CSF-1Rs.

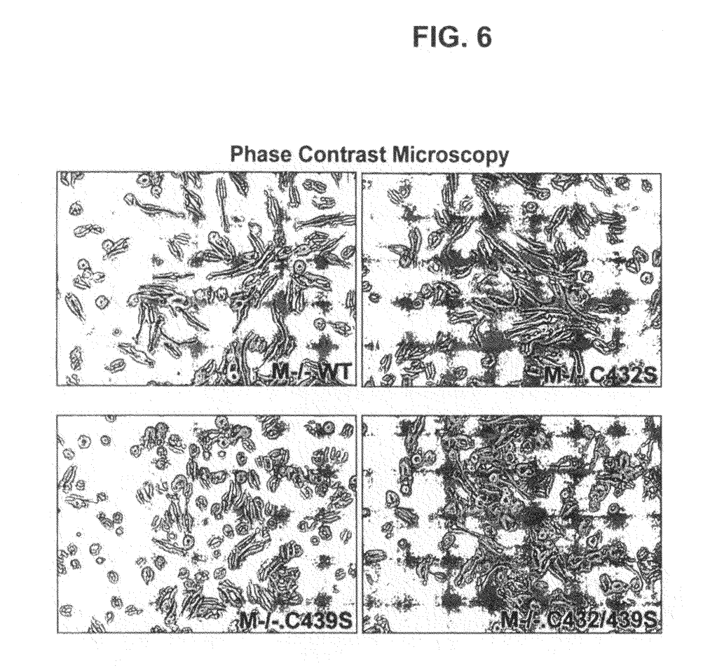

FIG. 6 shows the M−/− CSF-1R cell lines microscopically. M−/−.C439SCSF-1R and M−/−.C432/439SCSF-1R cells exhibit a less polarized morphology than M−/−.WTCSF-1R cells when cultured in the presence of human CSF-1.

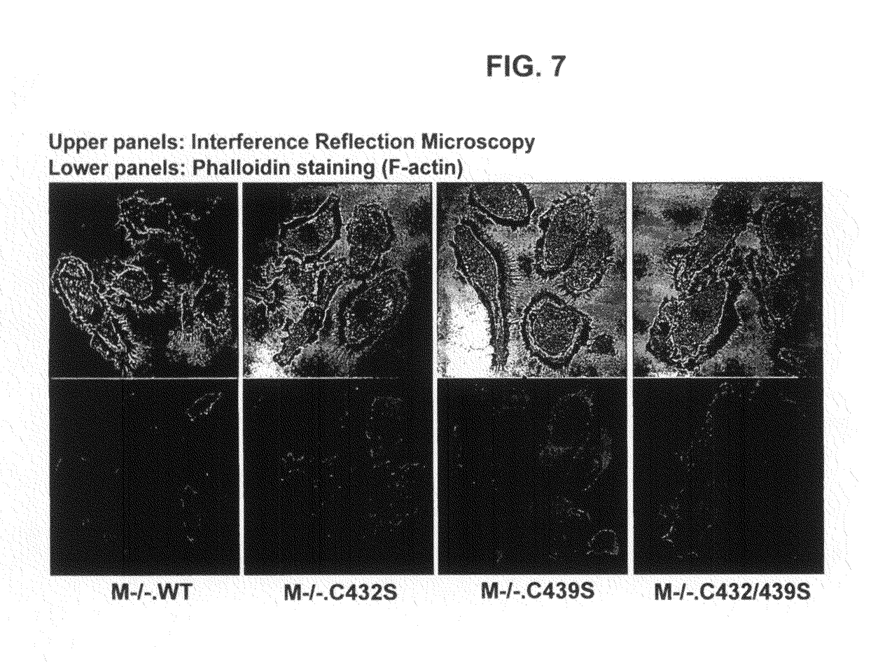

The cells were also examined using interference reflection microscopy and phalloidin staining, which stains F-actin. As shown in FIG. 7, M−/−.C439SCSF-1R and M−/−.C432/439SCSF-1R cells are more spread with fewer filopodia than M−/−.WTCSF-1R cells when cultured in the presence of human CSF-1.

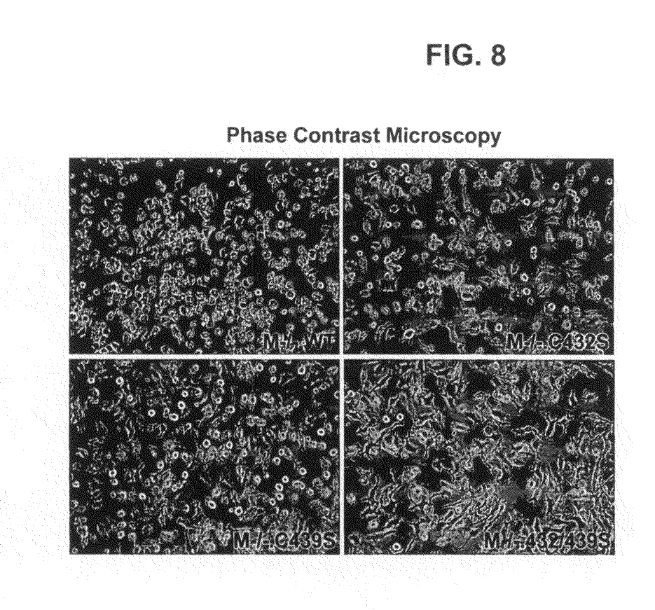

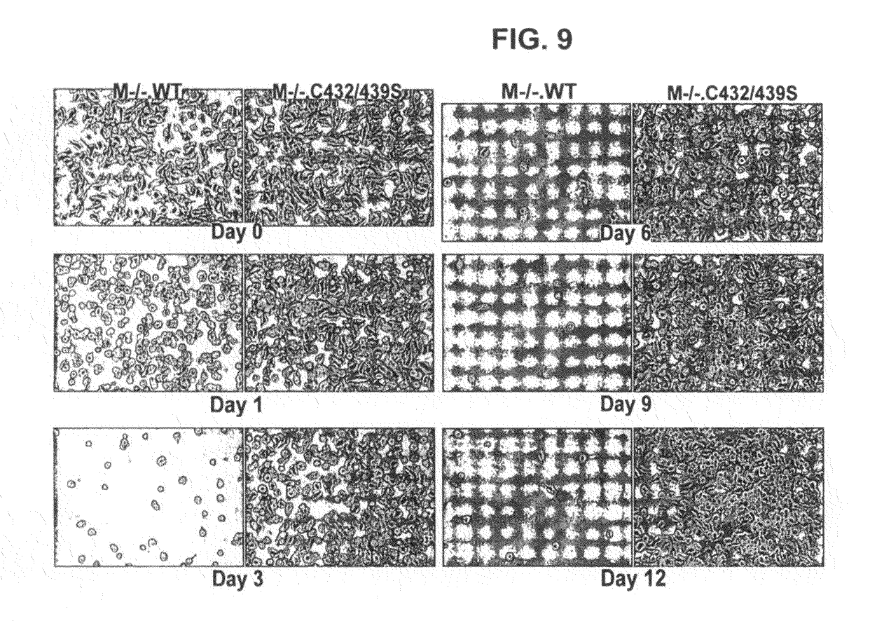

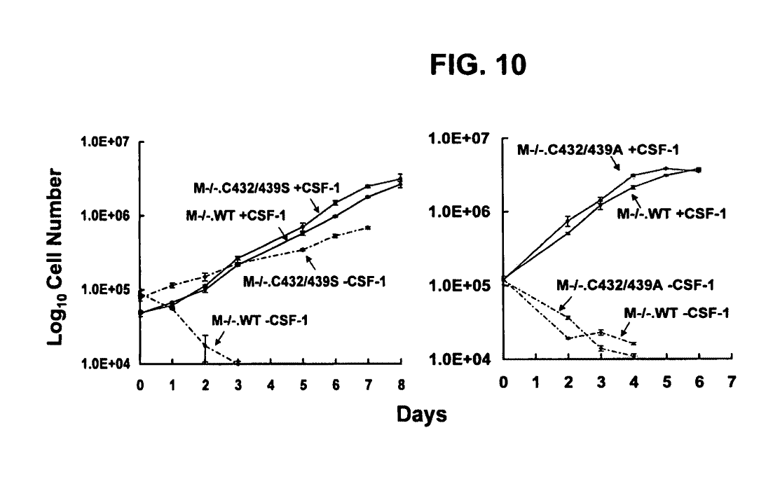

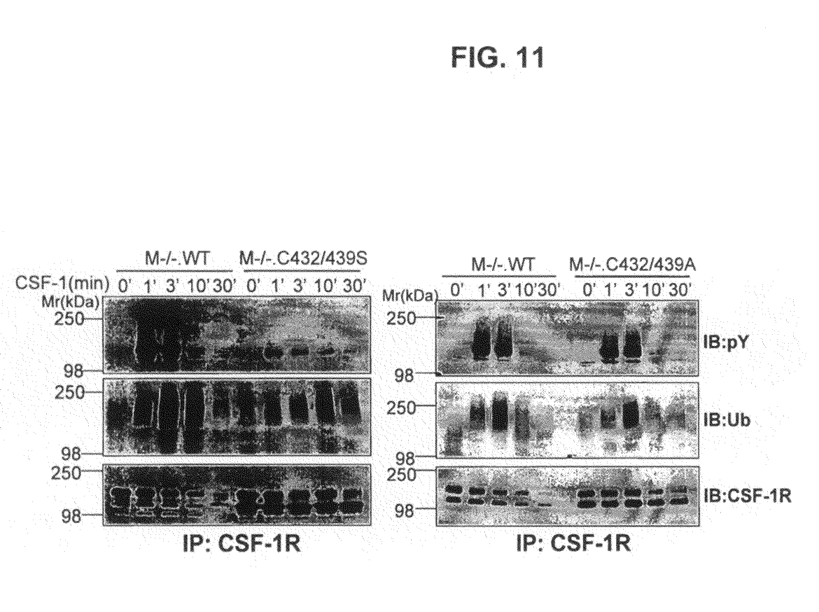

CSF-1 was removed from the cultures for 24 hours and the cultures were observed microscopically. As shown in FIG. 8, M−/−. C432SCSF-1R, M−/−.C439SCSF-1R and M−/−.C432/439SCSF-1R cells are more resistant to rounding up at 24 h after removal of CSF-1 than M−/−.WTCSF-1R cells. Observations were also made at Day 3, Day 6, Day 9 and Day 12. Compared with M−/−.WTCSF-1R cells, M−/−.C432/439SCSF-1R cells exhibit prolonged survival after removal of human CSF-1 (FIG. 9). Cell growth was quantified in these cultures (FIG. 10). Compared with M−/−.WTCSF-1R and M−/−.C432/439ACSF-1R cells, which die, M−/−.C432/439SCSF-1R cells proliferate when cultured in the absence of human CSF-1. Further, as shown in FIG. 11, following addition of human CSF-1 after culture for 20 h in the absence of CSF-1, M−/−.C432/439SCSF-1R cells, but not M−/−.C432/439ACSF-1R cells, exhibit dramatically decreased CSF-1R tyrosine phosphorylation, CSF-1R ubiquitination and decreased CSF-1R degradation compared with M−/−.WTCSF-1R cells.

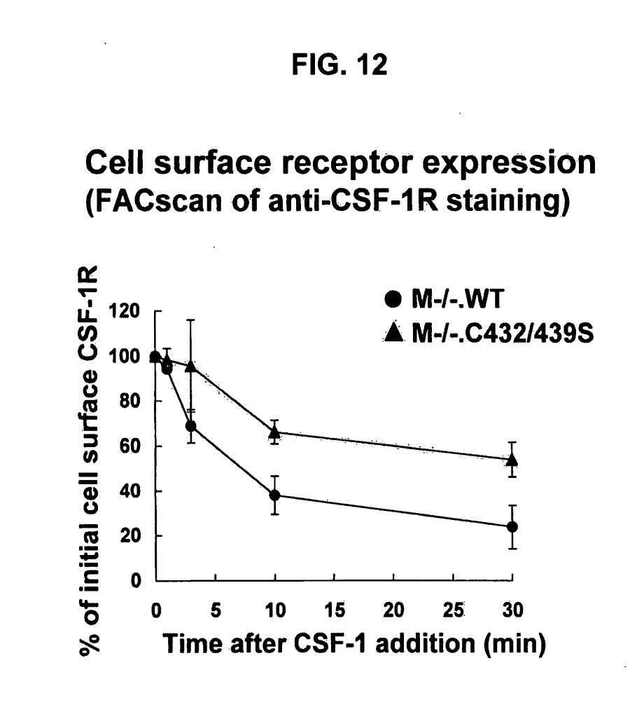

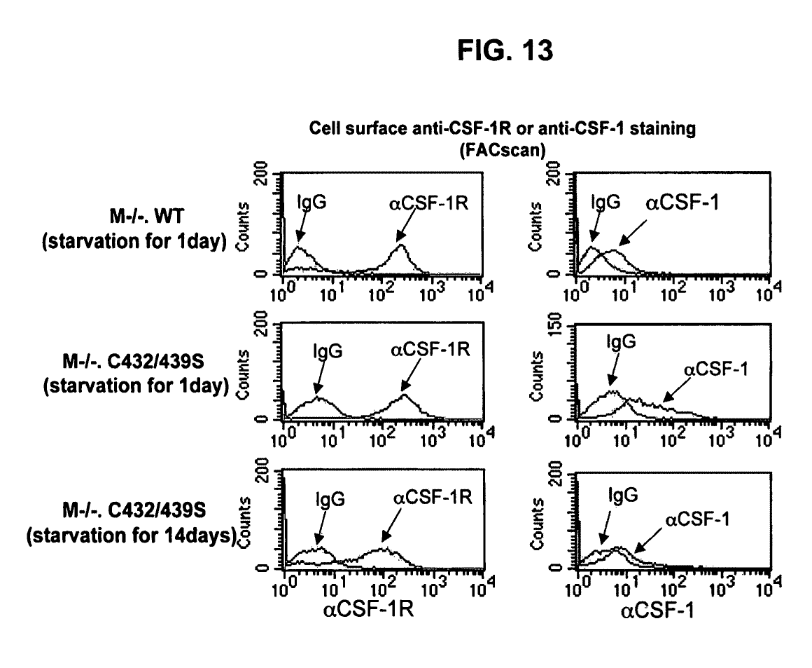

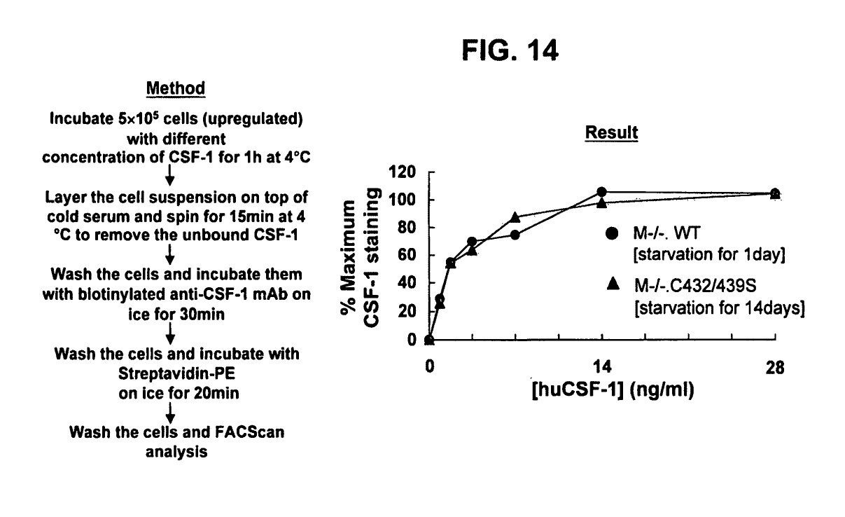

Cell surface receptor expression was evaluated next. As shown in FIG. 12, following culture for 20 h in the absence of CSF-1, the mutant CSF-1R in the M−/−.C432/439SCSF-1R cells exhibited slower internalization than the wild-type receptor in the M−/−.WTCSF-1R cells following addition of human CSF-1. In contrast to the absence of significant CSF-1 binding on the surface of M−/−.WTCSF-1R cells at 24 h after removal of human CSF-1, CSF-1 binding to the surface of M−/−.C432/439SCSF-1R cells persists, indicating that M−/−.C432/439SCSF-1R cells have a very slow CSF-1R off-rate (FIG. 13). Further, the initial rates of binding of CSF-1 to upregulated M−/−.WTCSF-1R and M−/−.C432/439SCSF-1R cells are indistinguishable (FIG. 14).

### Discussion

Mouse C432/439SCSF-1Rs bind human CSF-1 with normal kinetics but exhibit strikingly delayed CSF-1 dissociation rate and a lower rate of CSF-1-induced internalization and CSF-1R degradation. C432/439SCSF-1R cells starved of CSF-1 for 24 h still retain CSF-1 on their cell surface. In contrast to cells expressing the wt CSF-1R, after removal of CSF-1 they fail to exhibit a dramatic decrease in cell number, but continue to proliferate for up to 8 days. The CSF-1-induced tyrosine phosphorylation and ubiquitination of C432/439SCSF-1R cells starved of CSF-1 for 24 h is approximately 5-fold lower than cells expressing the wt CSF-1R, consistent with the majority of the mutant cell surface CSF-1Rs being occupied. These studies indicate that a soluble mouse CSF-1R ECD containing the C432/439S mutations should bind CSF-1 with a significantly higher affinity than the wt soluble CSF-1R ECD. Also preliminary evidence suggests that most of the effect of these mutations is mediated by the C439S mutation.

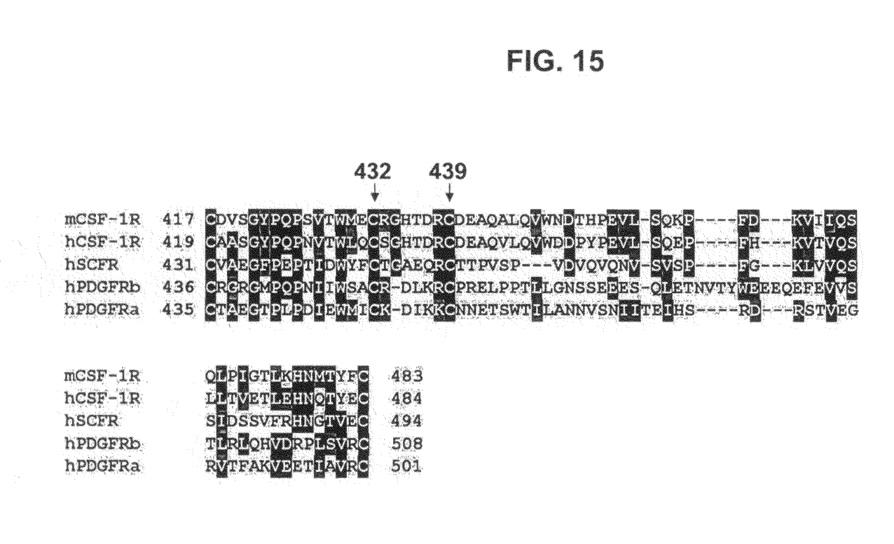

The mutations are in amino acids that are conserved in the human CSF-1R and in other class III receptor tyrosine kinases, including the stem cell receptor (SCFR) and the platelet dependent growth factor receptors alpha and beta (PDGFRα and PDGFRβ) (FIG. 15). Thus their introduction into a soluble form of the human CSF-1R (or other class III receptor) extracellular domain is expected to provide a competitive inhibitor of the binding of human CSF-1 (or other class III receptor ligand) that is much more effective than the wild type soluble CSF-1R (or other class III receptor) extracellular domain at inhibiting the action of CSF-1 (or other class III receptor ligand).

### REFERENCES

Aharinejad, S. et al., 2002, Cancer Res. 62:5317-5324.
Aharinejad, S. et al., 2004, Cancer Res. 64:5378-5384.
Berg K L, Siminovitch K A, Stanley E R, 1999, SHP-1 regulation of p62^{dok} tyrosine phosphorylation in macrophages. J Biol Chem 274:35855-65.
Chitu, V. and E. R. Stanley, 2006, Curr. Opin. Immunol. 18:39-48.
Dai, X-M., 2002, Blood 99:111-120.
Dey, A. et al., 2000, Mol. Biol. Cell 11:3835-3848.
Floege, J. et al., J. Am. Soc. Nephrol. 14:2690-2691.
Gandino L. and L. Varesio, 1990, Exp. Cell Res. 188:192-198.
Jat, P. S. and P. A. Sharp, 1986, J. Virol. 59:746-750.
Lennartsson, J. et al., 2005, Stem Cells 23:16-43.
Lotinun, S. et al., 2003; Endocrinol. 144:2000-2007.
Morgan, C. et al., 2005, J. Cell. Physiol. 130:420-427.
Muller A J, Young J C, Pendergast A M, Pondel M, Landau N R, Littman D R, Witte O N, 1991, BCR first exon sequences specifically activate the BCRJABL tyrosine kinase oncogene of Philadelphia chromosome-positive human leukemias. Mol Cell Biol 11:1785-92.
Paulus, P. et al., 2006, Cancer Res. 66:4349-4356.
Persons D A, Allay J A, Allay E R, Ashmun R A, Orlic D, Jane S M, Cunningham J M, Nienhuis A W, 1999, Enforced expression of the GATA-2 transcription factor blocks normal hematopoiesis. Blood 93:488-99.
Pirami, L. et al., 1991, Proc. Natl. Acad. Sci. USA 88:7543-7547.
Pixlet F. J. and E. R. Stanley, 2004, Trends in Cell Biol. 14:628-638.
Pollard, J. W. et al., 1991, Proc. Natl. Acad. Sci. USA 88:1474-1478.
Roberson S. M. and W. S. Walker, 1988, Cell Immunol. 116:341-351.
Rohde, C. M. et al., 2004, J. Biol. Chem. 42:43448-43461.
Roussel, M. F. et al., 1990, Proc. Natl. Acad. Sci. USA 87:6738-6742.
Sapi, E., 2004, Exp. Biol. Med. 229:1-11.
Schmid-Antomarchi, H. et al., 1998, Eur. Cytokine Netw. 9:99-108.
Shurtleff, S. A. et al., 1990, EMBO J. 9:2415-2421.
Stabel, J. R. and T. J. Stabel, 1995, Vet. Immunol. Immunopathol. 45:211-220.
Stanley E R, 1998, Macrophage Colony Stimulating Factor (CSF-1). In Delves P J, Roitt I M (eds): “Encyclopedia of Immunology.” Orlando: Academic Press, pp 1650-1654.
Stanley, E. R., 2000, pp. 911-934 In: Oppenheim J J and Feldman M, eds. Cytokine reference: A compendium of cytokines and other mediators of host defense. London: Academic Press.
Sudo T, Nishikawa S, Ogawa M, Kataoka H, Ohno N, Izawa A, Hayashi S I, Nishikawa S I, 1995, Functional hierarchy of c-kit and c-fms in intramarrow production of CFU-M. Oncogene 11:2469-2476.
Tushinski R J, Oliver I T, Guilbert L J, Tynan P W, Warner J R, Stanley E R, 1982, Survival of mononuclear phagocytes depends on a lineage-specific growth factor that the differentiated cells selectively destroy. Cell 28:71-81
Wilson, C. M. et al., 1991, J. Immunol. Methods 137:17-25.
Wrobel, C. N. et al., 2004, J. Cell Biol. 165:263-273.
Yeung, Y.-G. and E. R. Stanley, 2003, Mol. Cell. Proteomics 2:1143-1151.
   SEQ ID NOs SEQ ID NO:1. Wild-type mouse CSF-1R amino acid sequence, GenBank AAH36343. The two conserved cysteines mutated as described in the specification above is in bold underlined at residues 432 and 439.

| | |
|---|---|
| 1 | melgpplvll latvwhgqga pviepsgpel vvepgetvtl rcvsngsvew dgpispywtl |
| 61 | dpespgstlt trnatfkntg tyrcteledp magsttihly vkdpahswnl laqevtvveg |
| 121 | qeavlpclit dpalkdsysl mreggrqvlr ktvyffspwr gfiirkakvl dsntyvcktm |
| 181 | vngreststg iwlkvnrvhp eppqikleps klvrirgeaa qivcsatnae vgfnvilkrg |
| 241 | dtkleiplns dfqdnyykkv ralslnavdf qdagiyscva sndvgtrtat mnfqvvesay |
| 301 | lnltseqsll qevsvgdsli ltvhadayps iqhynwtylg pffedqrkle fitqraiyry |
| 361 | tfklflnrvk aseagqyflm aqnkagwnnl tfeltlrypp evsvtwmpvn gsdvlfcdvs |
| 421 | gypqpsvtwm ecrghtdrcd eaqalqvwnd thpevlsqkp fdkviiqsql pigtlkhnmt |
| 481 | yfckthnsvg nssqyfravs lgqskqlpde slftpvvvac msvmsllvll lllllykykq |
| 541 | kpkyqvrwki ieryegnsyt fidptqlpyn ekwefprnnl qfgktlgaga fgkvveataf |
| 601 | glgkedavlk vavkmlksta hadekealms elkimshlgq henivnllga cthggpvlvi |
| 661 | teyccygdll nflrrkaeam lgpslspgqd segdssykni hlekkyvrrd sgfssggvdt |
| 721 | yvemrpvsts ssdsffkqdl dkeasrplel wdllhfssqv aqgmaflask ncihrdvaar |
| 781 | nvlltsghva kigdfglard imndsnyvvk gnarlpvkwm apesifdcvy tvqsdvwsyg |
| 841 | illweifslg lnpypgilvn nkfyklvkdg yqmaqpvfap kniysimqsc wdleptrrpt |
| 901 | fqqicfllqe qarlerrdqd yanlpssggs sgsdsgggss ggsssepeee sssehlacce |
| 961 | pgdiaqpllq pnnyqfc |

SEQ ID NO:2. Wild-type human CSF-1R amino acid sequence, GenBank NP_{—}005202. The two conserved cysteines mutated as described in the specification above is in bold underlined at residues 434 and 441.

| | |
|---|---|
| 1 | mgpgvlllll vatawhgqgi pviepsvpel vvkpgatvtl rcvgngsvew dgppsphwtl |
| 61 | ysdgsssils tnnatfqntg tyrctepgdp lggsaaihly vkdparpwnv lagevvvfed |
| 121 | qdallpcllt dpvleagvsl vrvrgrplmr htnysfspwh gftihrakfi qsqdyqcsal |
| 181 | mggrkvmsis irlkvqkvip gppaltlvpa elvrirgeaa qivcsassvd vnfdvflqhn |
| 241 | ntklaipqqs dfhnnryqkv ltlnldqvdf qhagnyscva snvqgkhsts mffrvvesay |
| 301 | lnlsseqnli qevtvgegln lkvmveaypg lqgfnwtylg pfsdhqpepk lanattkdty |
| 361 | rhtftlslpr lkpseagrys flarnpggwr altfeltlry ppevsviwtf ingsgtllca |
| 421 | asgypqpnvt wlqcsghtdr cdeaqvlqvw ddpypevlsq epfhkvtvqs lltvetlehn |
| 481 | qtyecrahns vgsgswafip isagahthpp deflftpvvv acmsimalll lllllllyky |
| 541 | kqkpkyqvrw kiiesyegns ytfidptqlp ynekwefprn nlqfgktlga gafgkvveat |
| 601 | afglgkedav lkvavkmlks tahadekeal mselkimshl gqhenivnll gacthggpvl |
| 661 | viteyccygd llnflrrkae amlgpslspg qdpeggvdyk nihlekkyvr rdsgfssqgv |
| 721 | dtyvemrpvs tssndsfseq dldkedgrpl elrdllhfss qvaqgmafla skncihrdva |
| 781 | arnvlltngh vakigdfgla rdimndsnyi vkgnarlpvk wmapesifdc vytvqsdvws |
| 841 | ygillweifs lglnpypgil vnskfyklvk dgyqmaqpaf apkniysimq acwalepthr |
| 901 | ptfqqicsfl qeqaqedrre rdytnlpsss rsggsgssss eleeessseh ltcceqgdia |
| 961 | qpllqpnnyq fc |

SEQ ID NO:3. Wild-type human SCFR amino acid sequence, GenBank P05532. The two conserved cysteines mutated as described in the specification above is in bold underlined at residues 446 and 453.

| | |
|---|---|
| 1 | mrgargawdl lcvllvllrg qtatsqpsas pgepsppsih paqselivea gdtlsltcid |
| 61 | pdfvrwtfkt yfnemvenkk newiqekaea trtgtytcsn sngltssiyv fvrdpaklfl |
| 121 | vglplfgked sdalvrcplt dpqvsnysli ecdgkslptd ltfvpnpkag itiknvkray |
| 181 | hrlcvrcaaq rdgtwlhsdk ftlkvreaik aipvvsvpet shllkkgdtf tvvctikdvs |
| 241 | tsvnsmwlkm npqpqhiaqv khnswhrgdf nyerqetlti ssarvddsgv fmcyanntfg |
| 301 | sanvtttlkv vekgfinisp vknttvfvtd genvdlvvey eaypkpehqq wiymnrtsan |
| 361 | kgkdyvksdn ksniryvnql rltrlkgteg gtytflvsns dasasvtfnv yvntkpeilt |
| 421 | ydrlingmlq cvaegfpept idwyfctgae qrcttpvspv dvqvqnvsys pfgklvvqss |
| 481 | idssvfrhng tveckasndv gkssaffnfa fkeqiqahtl ftplligfvv aagamgiivm |
| 541 | vltykylqkp myevqwkvve eingnnyvyi dptqlpydhk wefprnrlsf gktlgagafg |
| 601 | kvveataygl iksdaamtva vkmlkpsahl terealmsel kvlsylgnhm nivnllgact |
| 661 | vggptivite yccygdllnf lrrkrdsfif skqeeqaeaa lyknllhste pscdssneym |
| 721 | dmkpgvsyvv ptktdkrrsa ridsyierdv tpaimeddel aldlddllsf syqvakamaf |
| 781 | laskncihrd laarnillth gritkicdfg lardirndsn yvvkgnarlp vkwmapesif |
| 841 | scvytfesdv wsygiflwel fslgsspypg mpvdskfykm ikegfrmvsp ehapaemydv |
| 901 | mktcwdadpl krptfkqvvq liekqisdst khiysnlanc npnpenpvvv dhsvrvnsvg |
| 961 | ssasstqpll vheda |

SEQ ID NO:4. Wild-type human PDGFβ amino acid sequence, GenBank NP_{—}002600. The two conserved cysteines mutated as described in the specification above is in bold underlined at residues 451 and 457.

| | |
|---|---|
| 1 | mrlpgampal alkgelllls lllllepqis qglvvtppgp elvinvsstf vltcsgsapv |
| 61 | vwermsqepp qemakaqdgt fssvltltnl tgldtgeyfc thndsrglet derkrlyifv |
| 121 | pdptvgflpn daeelfiflt eiteitipcr vtdpqlvvtl hekkgdvalp vpydhqrgfs |
| 181 | gifedrsyic kttigdrevd sdayyvyrlq vssinvsvna vqtvvrqgen itlmcivign |
| 241 | evvnfewtyp rkesgrlvep vtdflldmpy hirsilhips aeledsgtyt cnvtesvndh |
| 301 | qdekainitv vesgyvrllg evgtlqfael hrsrtlqvvf eayppptvlw fkdnrtlgds |
| 361 | sageialstr nvsetryvse ltivrvkvae aghytmrafh edaevqlsfq lqinvpvrvl |
| 421 | elseshpdsg eqtvrcrgrg mpqpniiwsa crdlkrcpre lpptllgnss eeesqletnv |
| 481 | tyweeeqefe vvstlrlqhv drplsvrctl rnavgqdtqe vivvphslpf kvvvisaila |
| 541 | lvvltiisli ilimlwqkkp ryeirwkvie syssdgheyi yvdpmqlpyd stwelprdql |
| 601 | vlgrtlgsga fgqvveatah glshsqatmk vavkmlksta rssekqalms elkimshlgp |
| 661 | hlnvvnllga ctkggpiyii teycrygdlv dylhrnkhtf lqhhsdkrrp psaelysnal |
| 721 | pvglplpshv sltgesdggy mdmskdesvd yvpmldmkgd vkyadiessn ymapydnyvp |
| 781 | sapertcrat linespvlsy mdlvgfsyqv angmeflask ncvhrdlaar nvlicegklv |
| 841 | kicdfglard imrdsnyisk gstflplkwm apesifnsly ttlsdvwsfg illweiftlg |
| 901 | gtpypelpmn eqfynaikrg yrmaqpahas deiyeimqkc weekfeirpp fsqlvlller |
| 961 | llgegykkky qqvdeeflrs dhpailrsqa rlpgfhglrs pldtssvlyt avqpnegdnd |
| 1021 | yiiplpdpkp evadegpleg spslasstln evntsstisc dsplepqdep epepqlelqv |
| 1081 | epepeleqlp dsgcpaprae aedsfl |

SEQ ID NO:4. Wild-type human PDGFα amino acid sequence, GenBank NP_{—}006197. The two conserved cysteines mutated as described in the specification above is in bold underlined at residues 450 and 456.

| | |
|---|---|
| 1 | mgtshpaflv lgclltglsl ilcqlslpsi lpnenekvvq lnssfslrcf gesevswqyp |
| 61 | mseeessdve irneennsgl fvtvlevssa saahtglytc yynhtqteen elegrhiyiy |
| 121 | vpdpdvafvp lgmtdylviv edddsaiipc rttdpetpvt lhnsegvvpa sydsrqgfng |
| 181 | tftvgpyice atvkgkkfqt ipfnvyalka tseldlemea lktvyksget ivvtcavfnn |
| 241 | evvdlqwtyp gevkgkgitm leeikvpsik lvytltvpea tvkdsgdyec aarqatrevk |
| 301 | emkkvtisvh ekgfieikpt fsqleavnlh evkhfvvevr aypppriswl knnltlienl |
| 361 | teittdveki qeiryrsklk lirakeedsg hytivaqned avksytfell tqvpssildl |
| 421 | vddhhgstgg qtvrctaegt plpdiewmic kdikkcnnet swtilannvs niiteihsrd |
| 481 | rstvegrvtf akveetiavr claknllgae nrelklvapt lrseltvaaa vlvllvivii |
| 541 | slivlvviwk qkpryeirwr viesispdgh eyiyvdpmql pydsrwefpr dglvlgrvlg |
| 601 | sgafgkvveg tayglsrsqp vmkvavkmlk ptarssekqa lmselkimth lgphlnivnl |
| 661 | lgactksgpi yiiteycfyg dlvnylhknr dsflshhpek pkkeldifgl npadestrsy |
| 721 | vilsfenngd ymdmkqadtt qyvpmlerke vskysdigrs lydrpasykk ksmldsevkn |
| 781 | llsddnsegl tlldllsfty qvargmefla skncvhrdla arnvllaqgk ivkicdfgla |
| 841 | rdimhdsnyv skgstflpvk wmapesifdn lyttlsdvws ygillweifs lggtpypgmm |
| 901 | vdstfynkik sgyrmakpdh atsevyeimv kcwnsepekr psfyhlseiv enllpgqykk |
| 961 | syekihldfl ksdhpavarm rvdsdnayig vtykneedkl kdweggldeq rlsadsgyii |
| 1021 | plpdidpvpe eedlgkrnrh ssqtseesai etgsssstfi kredetiedi dmmddigids |
| 1081 | sdlvedsfl |

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A mutant class III receptor tyrosine kinase (RTKIII) comprising a mutation at the amino acid residue corresponding to the conserved cysteine at residue 432 (C432) or 439 (C439) of a mouse colony-stimulating factor 1 receptor (CSF-1R) precursor having the amino acid sequence of SEQ ID NO:1, wherein the mutation is a replacement of the cysteine with an amino acid having an uncharged polar R group.

2. The mutant RTKIII of claim 1, wherein the replacement amino acid is a serine, a threonine, a tyrosine, an asparagine or a glutamine.

3. The mutant RTKIII of claim 1, wherein the replacement amino acid is a serine.

4. The mutant RTKIII of claim 1, wherein the mutation is a replacement of the cysteine at residue 432 with serine (C432S).

5. The mutant RTKIII of claim 1, wherein the mutation is C439S.

6. The mutant RTKIII of claim 1, comprising both C432S and C439S mutations.

7. The mutant RTKIII of claim 1, which has a delayed dissociation rate from its cytokine ligand when compared to the unmutated receptor.

8. The mutant RTKIII of claim 1, wherein the RTKIII is a CSF-1R, a platelet-derived growth factor receptor (PDGF-R), a stem cell factor receptor (SCFR), or a vascular endothelial growth factor receptor (VEGF-R).

9. The mutant RTKIII of claim 8, wherein the RTKIII is a CSF-1R.

10. The mutant RTKIII of claim 9, wherein the CSF-1R has a sequence at least 90% identical to SEQ ID NO:1 or SEQ ID NO:2.

11. The mutant RTKIII of claim 1, wherein the RTKIII is a human RTKIII.

12. The mutant RTKIII of claim 9, wherein the CSF-1R is a human CSF-1R.

13. The mutant RTKIII of any claim 1, wherein the RTKIII is a mouse RTKIII.

14. The mutant RTKIII of claim 9, wherein the RTKIII is a mouse CSF-1R.

15. The mutant RTKIII of claim 1, further comprising at least one other mutation from the wild-type RTKIII.

16. The mutant RTKIII of claim 14, wherein the mouse CSF-1R has the sequence of SEQ ID NO:1 except for the C432S and/or C439S mutation.

17. The mutant RTKIII of claim 12, wherein the human CSF-1R has the sequence of SEQ ID NO:2 except for a C434S and/or C441S mutation.

18. An extracellular domain of the mutant RTKIII of claim 1.

19-24. (canceled)

25. A stable cell line of macrophages lacking a native CSF-1R.

26-48. (canceled)

49. A method of preparing a stable cell line of macrophages from a mammal wherein the stable cell line maintains CSF-1 responsiveness, the method comprising
isolate macrophages from the mammal;
culture the macrophages in growth medium comprising CSF-1;
immortalize the macrophages using an SV-U19-5 retrovirus;
single cell plate the macrophages to create a cloned cell line; and
culture the cloned cell line to create the stable cell line.

50-57. (canceled)

58. A method of treating a mammal having or at risk for undesirable activation of a native RTKIII, the method comprising administering to the mammal the extracellular domain of claim 18, wherein the extracellular domain is from the same type of RTKIII as the native RTKIII.

59-75. (canceled)


SEQUENCE LISTING (5 sequences)

SEQ ID NO: 1 - protein, 977 aa, mouse
MELGPPLVLLLATVWHGQGAPVIEPSGPELVVEPGETVTLRCVSNGSVEWDGPISPYWTLDPESPGSTLTTRNATFKNTGTYRCTELEDPMAGSTTIHLYVKDPAHSWNLLAQEVTVVEGQEAVLPCLITDPALKDSVSLMREGGRQVLRKTVYFFSPWRGFIIRKAKVLDSNTYVCKTMVNGRESTSTGIWLKVNRVHPEPPQIKLEPSKLVRIRGEAAQIVCSATNAEVGFNVILKRGDTKLEIPLNSDFQDNYYKKVRALSLNAVDFQDAGIYSCVASNDVGTRTATMNFQVVESAYLNLTSEQSLLQEVSVGDSLILTVHADAYPSIQHYNWTYLGPFFEDQRKLEFITQRAIYRYTFKLFLNRVKASEAGQYFLMAQNKAGWNNLTFELTLRYPPEVSVTWMPVNGSDVLFCDVSGYPQPSVTWMECRGHTDRCDEAQALQVWNDTHPEVLSQKPFDKVIIQSQLPIGTLKHNMTYFCKTHNSVGNSSQYFRAVSLGQSKQLPDESLFTPVVVACMSVMSLLVLLLLLLLYKYKQKPKYQVRWKIIERYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDSEGDSSYKNIHLEKKYVRRDSGFSSQGVDTYVEMRPVSTSSSDSFFKQDLDKEASRPLELWDLLHFSSQVAQGMAFLASKNCIHRDVAARNVLLTSGHVAKIGDFGLARDIMNDSNYVVKGNARLPVKWMAPESIFDCVYTVQSDVWSYGILLWEIFSLGLNPYPGILVNNKFYKLVKDGYQMAQPVFAPKNIYSIMQSCWDLEPTRRPTFQQICFLLQEQARLERRDQDYANLPSSGGSSGSDSGGGSSGGSSSEPEEESSSEHLACCEPGDIAQPLLQPNNYQFC

SEQ ID NO: 2 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 3 - protein, 975 aa, Homo sapiens
MRGARGAWDLLCVLLVLLRGQTATSQPSASPGEPSPPSIHPAQSELIVEAGDTLSLTCIDPDFVRWTFKTYFNEMVENKKNEWIQEKAEATRTGTYTCSNSNGLTSSIYVFVRDPAKLFLVGLPLFGKEDSDALVRCPLTDPQVSNYSLIECDGKSLPTDLTFVPNPKAGITIKNVKRAYHRLCVRCAAQRDGTWLHSDKFTLKVREAIKAIPVVSVPETSHLLKKGDTFTVVCTIKDVSTSVNSMWLKMNPQPQHIAQVKHNSWHRGDFNYERQETLTISSARVDDSGVFMCYANNTFGSANVTTTLKVVEKGFINISPVKNTTVFVTDGENVDLVVEYEAYPKPEHQQWIYMNRTSANKGKDYVKSDNKSNIRYVNQLRLTRLKGTEGGTYTFLVSNSDASASVTFNVYVNTKPEILTYDRLINGMLQCVAEGFPEPTIDWYFCTGAEQRCTTPVSPVDVQVQNVSVSPFGKLVVQSSIDSSVFRHNGTVECKASNDVGKSSAFFNFAFKEQIQAHTLFTPLLIGFVVAAGAMGIIVMVLTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTVGGPTLVITEYCCYGDLLNFLRRKRDSFIFSKQEEQAEAALYKNLLHSTEPSCDSSNEYMDMKPGVSYVVPTKTDKRRSARIDSYIERDVTPAIMEDDELALDLDDLLSFSYQVAKAMAFLASKNCIHRDLAARNILLTHGRITKICDFGLARDIRNDSNYVVKGNARLPVKWMAPESIFSCVYTFESDVWSYGIFLWELFSLGSSPYPGMPVDSKFYKMIKEGFRMVSPEHAPAEMYDVMKTCWDADPLKRPTFKQVVQLIEKQISDSTKHIYSNLANCNPNPENPVVVDHSVRVNSVGSSASSTQPLLVHEDA

SEQ ID NO: 4 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 5 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL